(19) United States
(12) Patent Application Publication
(10) Pub. No.: US 2009/0285855 A1
(43) Pub. Date: Nov. 19, 2009

(54) RECOMBINANT VACCINE AGAINST JAPANESE ENCEPHALITIS VIRUS (JEV) INFECTION AND A METHOD THEREOF

(75) Inventor: Sudhanshu VRATI, New Delhi (IN)

Correspondence Address: LADAS & PARRY LLP, 26 WEST 61ST STREET, NEW YORK, NY, 10023, US

(73) Assignee: NATIONAL INSTITUTE OF IMMUNOLOGY

(21) Appl. No.: 12/426,306

(22) Filed: Apr. 20, 2009

Related U.S. Application Data
(62) Division of application No. 10/585,042, filed on Mar. 7, 2007.

(30) Foreign Application Priority Data
Jan. 4, 2004 (IN) 1516/DEL/2003

(51) Int. Cl. A61K 39/12; A61P 43/00

(52) U.S. Cl. 424/218.1

(57) ABSTRACT

The present invention relates to a novel recombinant adenovirus (RAdEs) vaccine against Japanese encephalitis virus (JEV) infection, wherein said vaccine produces secretory JEV envelope protein; an effective and superior method of immunization to JEV infection; also, a method of preparing the recombinant RAdEs vaccine.

### FIELD OF THE INVENTION

The present invention relates to a method of preparing a novel recombinant adenovirus (RAdEs) vaccine to protect against Japanese encephalitis virus (JEV) infection. Also, it relates to a method of immunization and to the vaccine per se.

### BACKGROUND AND PRIOR ART REFERENCES OF THE INVENTION

Japanese encephalitis virus (JEV) is a member of the flaviviridae family of animal viruses that consists of several viruses of immense medical significance such as those causing dengue and yellow fever. JEV, transmitted to human beings by mosquitoes, is responsible for an acute infection of the central nervous system resulting in encephalitis. The virus is active over a vast geographic area covering India, China, Japan and virtually all of the South-East Asia. Approximately 3 billion people live in JEV endemic area and up to 50,000 cases of JEV infection are reported every year, of which, about 10,000 cases result in fatality and a high proportion of survivors end up having serious neurological and psychiatric sequel (45). A mouse brain-grown, formalin-inactivated JEV vaccine is available internationally. However, this vaccine has limitations in terms of its high cost of production, lack of long-term immunity and risk of allergic reaction due to the presence of the murine encephalogenic basic proteins or gelatin stabilizer (1, 33, 38, 39). There is, thus, an urgent need to develop an improved vaccine against JEV and several potential vaccines are currently being investigated in various laboratories (16).

JEV contains a single-stranded, plus-sense RNA genome of ˜11 Kb. It consists of a single open reading frame that codes for a large polyprotein of 3432 amino acids which is co- and post-translationally cleaved into three structural (capsid, C; pre-membrane, prM; and envelope, E) and seven non-structural proteins (NS1, NS2A, NS2B, NS3, NS4A, NS4B and NS5) (5, 44). In flaviviruses, E protein is involved in a number of important functions related to virus infection such as receptor binding and membrane fusion (26). Antibodies to the E protein were shown to neutralize virus activity in vitro as well as if vivo since passive administration of monoclonal antibodies to the E protein protected mice with JEV infection (17). Furthermore, sub-viral particles consisting of only the prM and the E proteins were highly effective in generating protective immune response in mice against JEV (18, 25). Chen et al. (7) examined the potential of various JEV structural and non-structural proteins for vaccine development. They concluded that the E protein was the single most important protein capable of inducing protective immunity against JEV. Accordingly, several plasmid vectors have been described synthesizing different forms of JEV E protein with or without prM protein and these provided protection of varying degree in mice against Japanese encephalitis (2, 6, 15, 20).

In recent years, adenoviruses have shown great promise as vectors for recombinant vaccine development (3, 4, 8, 13, 35, 41, 42, 49). Besides being safe, these viruses have been shown to induce effective humoral and cellular immune responses in experimental animals when delivered orally, intramuscular (IM), intra-peritoneal or intra-nasal (3, 21, 24, 40, 41, 50). In the present invention, we constructed RAdEa expressing the prM and the full length JEV E protein since it is known to induce neutralizing immune response. However, adenovirus recombinant (RAdEa) synthesizing the full-length protein (Ea) did not grow well. Besides it induced poor immune response and very little JEV neutralizing antibodies. The full-length E protein is membrane anchored and removing the anchor signal from it is likely to make it soluble and perhaps more immunogenic.

On this premise, we truncated the Ea protein to remove 102-amino acid hydrophobic sequence from the C-terminus of the protein to generate a 398-amino acid Es protein. Recombinant adenovirus RAdEs synthesizing JEV prM and Es was found to grow very well in cultured cells and synthesize JEV E protein that was secreted into the medium. So, the removal of 102-amino acid hydrophobic sequence from the C-terminus of the protein played a critical role in smooth culturing of the recombinant. Even the inventors were pleasantly surprised with this effect.

Further, this recombinant adenovirus, synthesizing a novel form of JEV E protein that was secretory as opposed to the anchored protein in the normal course, was highly immunogenic in mice. RAdEs induced high titers of JEV neutralizing antibodies and protected the immunized mice against lethal JEV challenge demonstrating its potential use as a vaccine against JEV infection. The highly immunogenic effect of the recombinant helped achieve the desired results, which were long awaited by the scientific community.

The infection caused by JEV is fatal in nature and absence of long-term immunity had added to the problem. The allergic reactions to the known vaccine had further compounded the problem. However, administration of the vaccine of instant invention is been found to be absolutely safe and free from adverse effects.

### OBJECTS OF THE INVENTION

The main object of the present invention is to develop a recombinant adenovirus (RAdEs) vaccine against JEV infection.

Another main object of the present invention is to develop an effective and superior method of immunization to Japanese encephalitis virus (JEV) infection.

Yet another object of the present invention is to develop a plasmid pAdEs of SEQ ID No. 1.

Still another object of the present invention is to develop a method of preparing a recombinant adenovirus (RAdEs) vaccine to protect against Japanese encephalitis virus (JEV) infection.

### SUMMARY OF THE INVENTION

The present invention relates to development of a novel recombinant adenovirus (RAdEs) vaccine against Japanese encephalitis virus (JEV) infection.

### DETAILED DESCRIPTION OF THE INVENTION

Accordingly, the present invention relates to a novel recombinant adenovirus (RAdEs) vaccine against JEV infection; an effective and superior method of immunization to Japanese encephalitis virus (JEV) infection; also, a plasmid pAdEs of SEQ ID No. 1; and lastly, a method of preparing the recombinant adenovirus (RAdEs) vaccine.

In the main embodiment of the present invention, it relates to a method of preparing a recombinant adenovirus (RAdEs) vaccine to protect against Japanese encephalitis virus (JEV) infection, wherein the said vaccine produces secretory envelop protein of JEV, said method comprising steps of:
digesting plasmid pMEs with restriction enzymes Kpn I and Bam HI to obtain cDNA encoding JEV proteins prM and Es,
ligating the cDNA to adenovirus shuttle plasmid pShuttle digested with restriction enzymes Kpn I and Hind III at the Kpn I end,
filling nucleotides at the free Bam HI and Hind III ends with T4 DNA polymerase to create blunt ends,
ligating the blunt ends together to yield shuttle plasmid pSEs with JEV cDNA encoding the proteins prM and Es,
digesting the shuttle plasmid pSEs with restriction enzymes I-Ceu I and Pl-Sce I to obtain expression cassette containing the JEV cDNA together with the CMV promoter/enhancer and BGH polyadenylation signal,
ligating the digested shuttle plasmid with I-Ceu I and Pl-Sce I digested adenovirus plasmid pAdeno-X to generate plasmid pAdEs containing the Es expression cassette,
digesting the plasmid pAdEs with Pac I,
transfecting the monolayers of HEK 293 cells with digested plasmid pAdEs for about one week, and
obtaining the recombinant virus RAdEs vaccine.

In another embodiment of the present invention, the transfection is at about 37° C. temperature.

In yet another embodiment of the present invention, the secretory proteins are under the control of human CMV IE promoter/enhancer.

In another main embodiment of the present invention, the invention relates to a recombinant adenovirus (RAdEs) vaccine.

In yet another embodiment of the present invention, the vaccine produces secretory envelop protein (Es) of JEV.

In still another embodiment of the present invention, the vaccine protects against Japanese encephalitis virus (JEV) infection.

In still another embodiment of the present invention, the vaccine is effective by intramuscular route of administration.

In another main embodiment of the present invention, the invention relates to a plasmid pAdEs of SEQ ID No. 1.

In another main embodiment of the present invention, the invention relates to an effective and superior method of immunizing a subject in need thereof, to Japanese encephalitis virus (JEV) infection, said method comprising the step of administering a pharmaceutically effective amount of recombinant virus RAdEs vaccine optionally along with additive(s) to the subject intra-muscularly.

In yet another embodiment of the present invention, the method shows 100% efficacy.

In still another embodiment of the present invention, the method helps protect subject against Japanese encephalitis.

In still another embodiment of the present invention, the subject is animal.

In still another embodiment of the present invention, the subject is a human being.

In still another embodiment of the present invention, the immunization activates both humoral and cell-mediated immune responses.

In still another embodiment of the present invention, the humoral response to the vaccine is antibody IgG1 type.

In still another embodiment of the present invention, the method leads to high amount of IFN-gamma secretion.

In still another embodiment of the present invention, the immunization leads to IL-5 secretion at moderate levels.

In still another embodiment of the present invention, increased amounts of RAdEs lead to higher immune response.

In still another embodiment of the present invention, the method is more effective than the commercially available vaccine.

Replication-defective recombinant adenoviruses (RAds) were constructed that synthesized the pre-Membrane (prM) and envelope (E) proteins of Japanese encephalitis virus (JEV). Recombinant virus RAdEa synthesized Ea, the membrane-anchored form of the E protein, and RAdEs synthesized Es, the secretory E protein. RAdEa replicated poorly in human embryonic kidney (HEK) 293A cells and 88-folds lower titers of the virus were obtained compared to RAdEs. RAdEa also synthesized lower amounts of E protein in HEK 293A cells as judged by radioimmunoprecipitation and immunofluorescence studies.

Mice were immunized intramuscular (IM) and orally with RAds. Oral route of virus delivery induced low titers of anti-JEV antibodies that had only little JEV neutralizing activity. IM immunizations with both RAdEa and RAdEs resulted in high titers of anti-JEV antibodies. Interestingly, RAdEa induced very low titers of JEV neutralizing antibodies whereas RAdEs inoculation resulted in high titers of JEV neutralizing antibodies. Splenocytes from mice immunized IM with RAds secreted large amounts of interferon-γ and moderate amounts of interleukin-5. These splenocytes also showed cytotoxic activity against JEV-infected cells. Mice immunized IM with RAdEs showed complete protection against the lethal dose of JEV given intra-cerebral.

### BRIEF DESCRIPTION OF THE ACCOMPANYING DRAWINGS

FIG. 1 shows Growth of RAds in HEK 293A cells. Monolayers of HEK 293A cells in 35-mm tissue culture dishes were infected with RAds at a multiplicity of infection (MOI) of 1 and incubated at 37° C. in 3 ml culture medium. At 6 hr interval the cell monolayers were lysed in the culture medium by three cycles of freeze-thawing. The cell lysate was centrifuged to remove the debris and the supernatant assayed for adenovirus titers on HEK 293A cells. Shown in the figure are virus titers at various time points.

FIG. 2 shows Synthesis of JEV proteins in HEK 293A cells infected with RAds. HEK 293A cells were infected with JEV or RAds at a MOI of 1. Cells were labelled 24 hr later by growth in medium containing ³⁵S-methionine and ³⁵S-cystine. After labeling, culture supernatant (CS) and cells were harvested separately. Cells were washed with PBS before the cell lysate (CL) was made. CLs and CSs were used for immunoprecipitation of JEV proteins with mouse anti-JEV serum. The proteins were separated on a 12% SDS-polyacrylamide gel and autoradiographed. Lanes containing proteins precipitated from JEV-, RAdEa- or RAdEs-infected or uninfected cells have been identified. Position of JEV proteins has been indicated at the left. Ea, Es and prM proteins synthesized by RAds have been indicated. The values on the right are molecular size markers in kDa.

FIG. 3 shows Immunofluorescent staining of virus-infected cells. HEK 293A cells were infected with RAds or JEV at a MOI of 1. The cells were fixed and permeabilized 24 hr later and stained with mouse anti-JEV serum followed by FITC-conjugated goat anti-mouse-IgG. They were then observed under a microscope using ultra-violet light. Panel A, RAdEa-infected cells; panel B, RAdEs-infected cells; panel C, JEV-infected cells and panel D, uninfected cells.

FIG. 4 shows Antibody response in mice. BALB/c mice were immunized with RAdEa, RAdEs or with the vaccine by IM or oral route of inoculation. The dosages of the immunogens (in PFU for RAds) and the routes of inoculation have been indicated below the figure. *indicates oral immunization where virus was diluted in 100 mM Sodium bicarbonate buffer (pH 8.9). The primary immunization was followed by booster doses that were given 21 and 36 days later. Mice were bled at day 20, 28 and 44 post-immunization and sera stored at −70° C. Serial two-fold dilutions of sera (starting at 1:25) were assayed for the end-point anti-JEV antibody titers by ELISA. Shown above are mean end-point titers of sera obtained from immunized mice as indicated below the figure. The open bars represent titers on day 20, the gray bars represent titers on day 28 and black bars show titers on day 44 post-immunization.

FIG. 5 shows Isotype analysis of anti-JEV antibody produced by immunized mice. BALB/c mice were immunized with RAdEa, RAdEs or with the vaccine by IM or oral route of inoculation. The dosages of the immunogens (in PFU for RAds) and the routes of inoculation have been indicated below the figure. The primary immunization was followed by booster doses given 21 and 36 days later. Mice sera obtained on day 44 post-immunization were assayed for the end-point ELISA titers of anti-JEV IgG1 and IgG2a antibodies. Serial two-fold dilutions of sera (starting at 1:25) were assayed for the end-point titers. The ELISA titer was recorded as zero if the 1:25 dilution of sample was negative in ELISA. Shown in the figure are mean end-point titers of sera obtained from immunized mice as indicated below the figure. The open bars represent IgG1 titers and the gray bars represent IgG2a titers. The inverted triangle indicates zero mean titer.

FIG. 6 shows JEV neutralizing antibody response in mice. BALB/c mice were immunized with RAdEa, RAdEs or the vaccine by IM or oral route of inoculation. The dosages of the immunogens and the route of inoculation have been indicated at the top of the figure. The primary immunization was followed by booster doses given 21 and 36 days later. *indicates oral immunization where virus was diluted in 100 mM Sodium bicarbonate buffer. Mice were bled on day 44 post-immunization and sera stored at −70° C. Serial two-fold dilutions of sera (starting at 1:10) were assayed for end-point JEV neutralization titers by plaque reduction neutralization assays. Shown in the figure are mean JEV neutralization titers of serum samples from immunized mice.

FIG. 7 shows Cytokine production by splenocytes from immunized mice. BALB/c mice were immunized with RAdEa, RAdEs or the vaccine by IM or oral route of inoculation as indicated at the top of the figure. These mice were given two booster doses as described in the methods. One week after the second booster dose splenocytes from two mice (indicated by gray and black bars) from each immunization group were cultured in presence of JEV. Culture supernatants were collected each day and stored at −70° C. These were then assayed for IFN-γ, IL-4 and IL-5. Shown in the figure are the levels of IFN-γ and IL-5 in splenocyte cultures on various days after incubation with JEV. The days are numbered at the bottom of the panel.

FIG. 8 shows CTL activity in immunized mice. BALB/c mice were immunized with RAdEa or RAdEs by IM route of inoculation. These mice were given two booster doses as described in the methods. One week after the second booster dose splenocytes from two mice (indicated by gray and black bars) from each immunization group were cultured in presence of JEV for the generation of effector cells. Shown in the figure is the CTL activity of splenocytes at various ratios of effector cells to target cells (E:T) which are indicated at the bottom of the panels.

FIG. 9 shows Mice survival after challenge. Groups of 6-8 BALB/c mice were immunized with various dosages of RAdEa and RAdEs through IM or oral route. These mice received two booster doses of the immunogen on day 22 and 36 post-immunization. Mice were challenged on day 44 post-immunization with 100 LD₅₀ (50%-lethal dose) of JEV given intra-cerebrally. Mice were observed for mortality for the next three weeks. Shown above is the percentage of surviving mice at a given time point. Immunogen dose and route of inoculation have been indicated. *indicates oral delivery of RAdEs along with the bicarbonate buffer.

The invention is further elaborated with the help of experimental data, as presented below in the form of examples. However, the examples should not be construed to limit the scope of the invention

### EXAMPLE-1

JEV and cells: The GP78 strain of JEV was used in these studies (44). The virus was grown in neonatal mouse brain. The brain from infected mice was homogenized as a 10% suspension in Eagle's minimal essential medium (EMEM). The suspension was centrifuged and filtered through 0.22 μm sterile filters. The virus was stored at −70° C. in aliquots. Virus titration was carried out by plaque assay on porcine stable kidney (PS) monolayers as described previously (43). Adenovirus was grown in human embryonic kidney (HEK) 293A cells (Quantum Biotechnologies Inc.) cultured in Dulbecco's modified Eagle's medium (DMEM) supplemented with 10% fetal calf serum (FCS).

### EXAMPLE-2

Construction of recombinant adenoviruses: Recombinant adenoviruses were constructed using the Adeno-X expression system (BD Biosciences) that utilized a ligation-based strategy for producing recombinant virus. Using this system a mammalian expression cassette containing the cDNA encoding JEV E protein was incorporated into a replication incompetent (E1/E3) human adenovirus type 5 (Ad5) genome. Two recombinant adenoviruses (RAds) were made; RAdEa, synthesizing JEV prM and the membrane-anchored E protein, and RAdEs, synthesizing prM and the secretory E protein. Plasmids pMEa and pMEs that contained the cDNAs encoding JEV prM and the membrane-anchored (Ea) or secretory E protein (Es), respectively have been described previously (15). Plasmid pMEa was modified by site-directed mutagenesis to contain an Afl II restriction site down-stream of the 3′-end of the JEV cDNA past the Bam HI site. The JEV cDNA encoding the prM and Ea proteins was excised from the mutated pMEa as a Kpn I-Afl II fragment and cloned at these sites in the adenovirus shuttle plasmid pShuttle (BD Biosciences) under the control of human cytomegalovirus (CMV) immediate early (IE) promoter/enhancer to yield plasmid pSEa. The shuttle plasmid pSEa contained the bovine growth hormone (BGH) polyadenylation signal downstream of the cloned JEV cDNA. The cDNA encoding prM and Es sequence was obtained by digesting pMEs with Kpn I and Bam HI. This cDNA fragment was ligated to the adenovirus shuttle plasmid, pShuttle, digested with Kpn I and Hind III at the Kpn I end. The free Bam HI and Hind III ends were nucleotide filled with T4 DNA polymerase and the blunt ends so created were ligated together to yield plasmid pSEs where JEV cDNA encoding prM and Es was under the control of human CMV IE promoter/enhancer. The shuttle plasmid pSEs contained the BGH polyadenylation signal downstream of the cloned cDNA. The junctions of the shuttle plasmid and the cDNA insert were sequenced to confirm the presence of the cDNA in correct frame. The plasmids pSEa and pSEs were digested with I-Ceu I and Pl-Sce I to obtain the expression cassettes containing the JEV cDNA together with CMV promoter/enhancer and BGH polyadenylation signal. These were then ligated with I-Ceu I and Pl-Sce I digested adenovirus plasmid pAdeno-X (BD Biosciences) to generate plasmids pAdEa and pAdEs containing the Ea and Es expression cassettes, respectively. Monolayers of HEK 293A cells were transfected with Pac I digested pAdEa and pAdEs using Effectene (Qiagen) and incubated for a week at 37° C. By this time, RAd plaques had begun to show. The cell monolayers were harvested in culture supernatant, frozen-thawed thrice and centrifuged to obtain the released crude recombinant virus that was amplified once in HEK 293A cells and subjected to 2 rounds of plaque purification to obtain recombinant viruses RAdEa and RAdEs.

### EXAMPLE-3

Radioimmunoprecipitation: Synthesis of JEV proteins by RAdEa and RAdEs was studied by infection of HEK 293A cells followed by radiolabelling and immunoprecipitation. Briefly, monolayers of HEK 293A cells were infected with virus at a multiplicity of infection (MOI) of 1 and incubated at 37° C. After 24 hr, the cell monolayer was radiolabelled by growing in presence of 100 μCi of Easytag™ EXpre³⁵S³⁵S protein labeling mix (NEN) for 4 hr. The culture supernatant was harvested and stored at −70° C. The monolayers were harvested in 500 μl of radioimmunoprecipitation assay buffer (10 mM Tris-HCl pH 8.0, 140 mM NaCl, 5 mM Iodoacetamide, 0.5% Triton X-100, 1% Sodium dodecyl sulphate (SDS), 1% Sodium deoxycholate, 2 mM Phenylmethylsulfonyl fluoride). Immunoprecipitation was carried out using mouse anti-JEV serum (ATCC) and Protein A Sepharose beads (Amersham). Immunoprecipitated proteins were electrophoresed on a 12% SDS-polyacrylamide gel that was dried before exposing to X-ray film for autoradiography.

### EXAMPLE-4

Immunofluorescent staining: Monolayers of HEK 293A cells were infected with RAdEa, RAdEs or JEV at a MOI of 1. The cells were fixed the next day with 2% Paraformaldehyde and permeabilized with 0.1% Triton-X 100. These cells were then stained by incubation with mouse anti-JEV serum (ATCC) followed by anti-mouse IgG-FITC conjugate (Dako) and observed under a microscope using ultra-violet light.

### EXAMPLE-5

Mice immunization and challenge experiments: All immunizations were carried out on 4-5-week-old inbred BALB/c mice. Each immunization group consisted of 6-8 mice. For IM immunizations, mice were injected in the hind legs with different amounts of RAds diluted in 100 μl phosphate-buffered saline (PBS) using a 30 G needle. Another group of mice was immunized with the mouse brain-derived, formalin-inactivated JEV vaccine manufactured by the Central Research Institute, Kasauli (India). Each mouse was given an IM injection of 100 μl of the vaccine that was 1/10^{th} of the recommended adult human dose. For oral immunizations, water was withdrawn from the mice cages for 6-8 hr and then 200-400 μl virus diluted in PBS or 100 mM Sodium bicarbonate buffer (ph 8.9) was administered per oral using a mouse-feeding needle that had a small balloon at the point of the delivery. Two booster doses, given 3 and 5 weeks after the primary immunization, contained the same amount of immunogen as the primary dose. At 44 days post-immunization, mice were challenged with intra-cerebral inoculation of the lethal dose of JEV. These mice were observed for mortality for the next 3 weeks.

### EXAMPLE-6

Antibody assays: Titers of anti-JEV antibodies were assayed using an enzyme-linked immunosorbent assay (ELISA). An ELISA plate was coated with C6/36 cell-grown JEV overnight at 4° C. in 0.2 M Sodium carbonate buffer, pH 9.6 (36). The plate was washed with PBS containing 0.1% Tween-20 (PBS-T) thrice and the wells were blocked with 1% Lactogen in PBS-T at 37° C. for 2 hr. The plate was again washed with PBS-T thrice and incubated with 100 μl diluted mice sera per well at 37° C. for 1 hr. Serial two-fold dilutions of sera were assayed starting at a dilution of 1:25. The plate was then washed thrice with PBS-T and 100-μl anti-mouse antibody conjugated to horse radish peroxidase (HRP) (Dako), diluted 1:2000, was added per well, followed by incubation at 37° C. for 1 hr. The antibody-conjugate was removed by washing the plate thrice with PBS-T. The plate was then incubated in dark with 100 μl per well of the substrate o-Phenylenediamine dihydrochloride (0.5 mg/ml) prepared in citrate buffer (1% Citric acid and 1.46% Disodium hydrogen phosphate) at room temperature for 10 min. The reaction was stopped by adding 50 μl of 5 N Sulfuric acid. The absorbance was read at 492 nm in an ELISA plate reader (Spectramax). The reciprocal of the highest serum dilution giving an optical density at least twice that given by the reagent blanks was taken as the ELISA end-point.

The antibody isotyping ELISAs were carried out in a similar fashion, except that in place of anti-mouse IgG-HRP conjugate, anti-mouse IgG1-HRP or the IgG2a-HRP conjugate (Pharmingen) was added. This was followed by the incubation with the substrate and color development as above.

### EXAMPLE-7

Plaque reduction neutralization assay: Two fold serial dilutions of sera from the immunized mice (starting from 1:10) were prepared in EMEM containing 5% FCS and antibiotics. Diluted sera were incubated at 56° C. for 30 min to inactivate the complement. The serum sample (100 μl) was then mixed with equal volume of JEV culture supernatant containing 100 plaque-forming units (PFU) of the virus. The virus-antibody mixture was incubated at 37° C. for 1 hr before adding to a 35-mm dish containing ˜70% confluent monolayer of PS cells. The plaque assay was carried out as described. (43). Percent neutralization was calculated by counting the number of plaques in the presence and the absence of the mouse serum. All assays were done in duplicates. Reciprocal of the highest serum dilution giving 50% neutralization was taken as the JEV neutralization titer.

### EXAMPLE-8

Cytokine ELISA: Spleen cell suspensions were prepared in RPMI 1640 supplemented with 10% FCS and 6×10⁷ splenocytes were incubated with 3×10⁷ PFU of JEV or E. coli-synthesized JEV E protein (10 μg/ml) in a 35-mm dish at 37° C. Aliquots of culture supernatant were removed every day for the next 4 days and stored at −70° C. Interleukin (IL)-4, IL-5 and interferon (IFN)-γ were assayed using BD OptEIA kits (BD Biosciences) and as per the assay protocols.

### EXAMPLE-9

Cytotoxic T lymphocyte (CTL) assay: Standard ⁵¹Cr-release method as described previously (15) was used for the CTL assays with some modifications. Briefly, for preparation of the responder cells, 3×10⁷ splenocytes were incubated for 4 days with 3×10⁷ PFU of JEV in a 35-mm dish in RPMI 1640 medium supplemented with antibiotics, 0.5 mM β-Mercaptoethanol, 0.32 mg/ml L-Glutamine, 0.1 mg/ml non-essential amino acids, 0.12 mg/ml Sodium pyruvate and 5% FCS at 37° C. The cells thus generated were referred to as the effector cells. Virus-infected target cells were prepared by infecting P388D1 cells with JEV for 48 hr at a MOI of 1 followed by incubation with 100 μCi of Na₂⁵¹CrO₄ (NEN) and subsequent washings to remove free ⁵¹Cr.

For carrying out the CTL assay, various numbers of effector cells were incubated at 37° C. for 6 hr with 2×10^{4 51}Cr-labelled virus-infected cells by briefly centrifuging them at 100 g for 4 min in a 96-well round bottom plate. At the end of the incubation period, 100 μl of cell-free supernatant was removed and the ⁵¹Cr release was counted using a gamma counter (LKB). Triplicate estimations were done in all the assays and the percentage lysis was calculated using the following formula. Percent lysis=[(cpm released in the presence of effector cells—cpm released due to spontaneous leakage)/(total cpm released by 0.2% Triton X-100 lysis—cpm released due to spontaneous leakage)]×100.

Statistical analysis: The statistical significance of different findings between mouse groups was determined by Student's t test. P≦0.05 was considered to be significant.

### EXAMPLE-10

Replication of RAds and synthesis of JEV proteins: The JEV E protein is 500 amino acids long membrane-anchored protein. We have earlier shown that deletion of the C-terminal 102 amino acids of JEV E protein leads to efficient secretion of the truncated protein into the cell surroundings (15). We had also shown that quality of immune responses in mice induced by the secretory E protein were different from those induced by the membrane-anchored E protein (15). Besides, vaccinia recombinants expressing the secretory E protein of Japanese encephalitis or Dengue viruses devoid of the membrane anchor sequence were found to be highly immunogenic in mice (14, 27, 37). We, therefore, constructed two recombinants, RAdEa synthesizing prM and Ea (full-length membrane-anchored JEV E protein) and RAdEs synthesizing prM and Es (398 amino acid secretory E protein). The JEV prM was included in our constructs as its co-synthesis was necessary for correct processing and folding of the E protein (18, 25). The presence of appropriate JEV cDNA in genomes of RAds was established by polymerase chain reaction using JEV genome sequence-specific oligonucleotide primers.

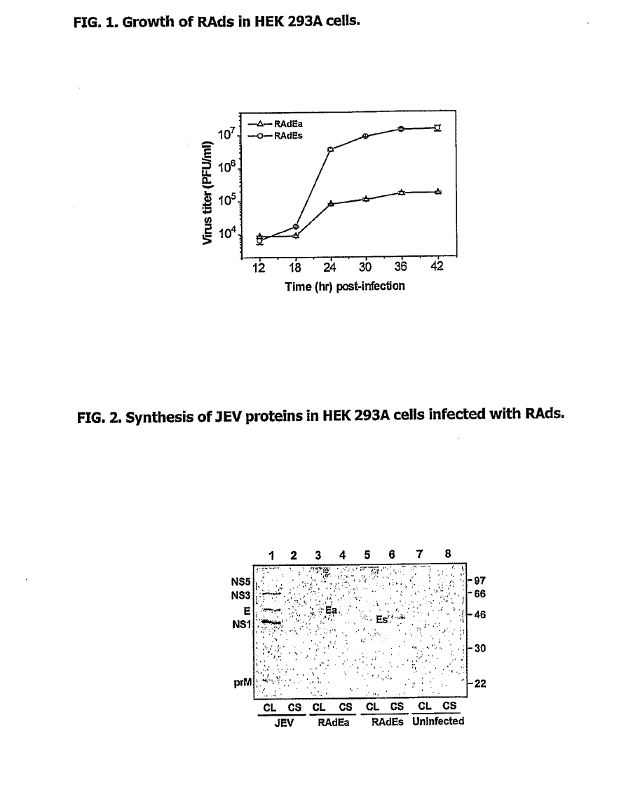

Replication of RAds was studied in HEK 293A cells infected at a MOI of 1. FIG. 1 shows that a major burst in viral titers was observed both for RAdEa and RAdEs between 18 and 24 hr post-infection (PI) after which there was only a marginal increase in titers. RAdEs titer was ˜40-fold higher than that of RAdEa at 24 hr PI. Following the virus replication, the cytopathic effects were first visible at 30 hr PI for both RAdEa and RAdEs. The cytopathic effects had become highly pronounced by 42 hr PI when almost 80% cells had come off the surface of the tissue culture plates. At this time point titer of RAdEs was 1.2×10⁷ PFU/ml, which was 88-times higher than that of RAdEa. These results showed that compared to RAdEs, RAdEa replicated poorly in HEK 293A cells. We studied four independent isolates of RAdEa and found all them to be slow growers.

The synthesis of JEV proteins by RAds was studied in HEK 293A cells that were infected with different viruses followed by radiolabelling and immunoprecipitation of JEV proteins using mouse anti-JEV serum. FIG. 2 shows that RAdEa-infected cell lysate had 2 proteins of apparent molecular masses of 51 and 23 kDa that corresponded with JEV E and prM proteins. None of these proteins were detectable in the culture supernatant of the infected cells. The RAdEs-infected cell lysate showed the synthesis of Es and prM proteins of apparent molecular masses of 45 and 23 kDa, respectively. The culture supernatant from the RAdEs-infected cells had a significant amount of Es, indicating that the E protein synthesized by RAdEs was secretory.

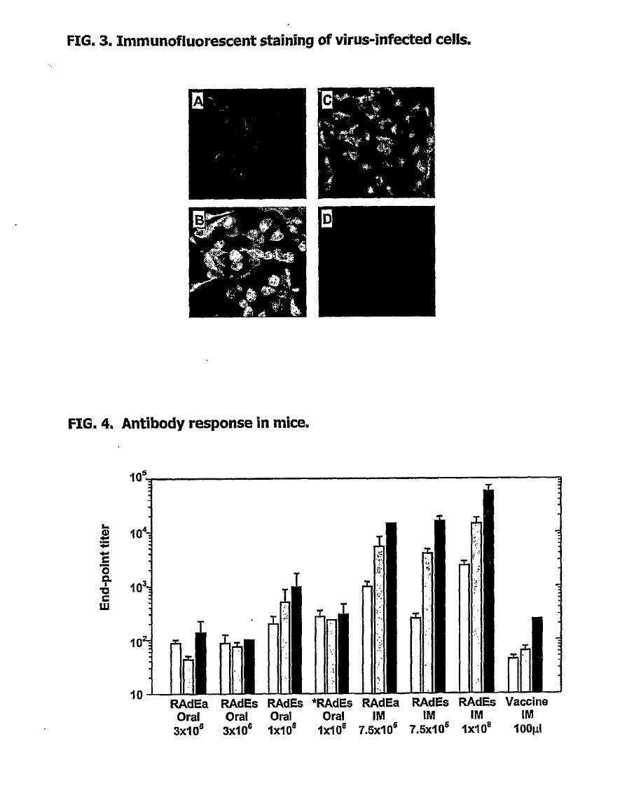

Use of increased amounts of anti-JEV antibody and Protein A Sepharose in immunoprecipitations did not result in precipitation of enhanced amounts of E protein indicating that the amounts of these reagents used were not limiting. Scanning of the autoradiograph with optically-enhanced densitometer using ‘Diversity One’ software (version 1.6, PDI, New York) followed by calculations based on the volumes of the lysate or the supernatant loaded on the gel, suggested that the secretory E protein present in the culture supernatant constituted about 76% of the total E protein synthesized by RAdEs-infected cells. Furthermore, levels of total Es protein synthesis were around 20-fold higher than the levels of Ea protein. Immunofluorescent staining of RAdEa- and RAdEs-infected HEK 293A cells further confirmed that levels of Ea protein were significantly lower than that of the Es protein (FIG. 3). The lower levels of Ea synthesis may be related to the poor growth of RAdEa in HEK 293A cells.

We found that RAdEa synthesizing the membrane-anchored E protein grew slowly and achieved 88-fold lower titers in HEK 293A cells when compared with RAdEs synthesizing the secretory E protein. Infection of HEK 293A cells with the recombinants followed by radioimmunoprecipitation of JEV proteins showed that RAdEa synthesized lower amounts of JEV E protein. This was corroborated by the low levels of immunofluorescence on RAdEa-infected HEK 293A cells when compared with RAdEs-infected cells. HEK 293A cells support replication of E1-deleted RAds reported in this work as these cells contain Ad5 E1 transcription unit permanently integrated in them. In other mammalian cells these RAds are unable to replicate for the want of the E1 sequences. However, expression of the foreign gene does take place, which is under the independent control of the CMV IE promoter. We have studied expression of JEV E protein by RAds in PS cells by immunofluorescence and radioimmunoprecipitation. No significant differences were found in the levels of E protein synthesis in PS cells infected with RAdEa or RAdEs at a MOI of 1.

The adenovirus vector used in our studies had deletions in both the E1 and E3 transcription units that allow packaging of up to 8 Kb foreign DNA in the recombinant virus. The size of JEV expression cassette inserted in RAdEa was ˜3.3 Kb which was well within the packaging limits of the recombinant virus. The reason for lower titers of RAdEa in HEK 293A cells is, thus, not clear. Previously, we found no differences in the levels of expression of membrane-anchored or secretory E protein of JEV when HEK 293A cells were transfected with expression plasmids containing the Ea or Es genes under CMV IE promoter (15). This observation together with our results in the PS cells on E protein synthesis indicates that lower levels of E protein synthesis by RAdEa in HEK 293A cells are related to the low levels of its replication.

### EXAMPLE-11

Anti-JEV antibody response in mice immunized with RAds: Groups of BALB/c mice were immunized with RAdEa or RAdEs delivered orally or IM. The antibody response of these mice was compared with those immunized with formalin-inactivated commercial JEV vaccine. Serum samples collected from mice at various time points were assayed for anti-JEV end-point ELISA titers. FIG. 4 shows that anti-JEV antibodies were detectable in all immunization groups on day 20 post-immunization and these titers increased further on days 28 and 44 post-immunization following the booster doses. Compared to IM immunization, antibody titers were drastically low in mice immunized orally with RAds. For example, compared to oral immunization, 1×10⁸ PFU of RAdEs given IM induced ˜60-fold higher antibody response on day 44. For both the oral as well as IM immunizations, higher antibody titers were obtained when higher doses of RAd were used. No significant differences were seen in day 44 anti-JEV antibody titers of mice immunized IM with 7.5×10⁵ PFU of RAdEa or RAdEs. IM immunizations with RAds induced significantly higher antibody titers than those induced by IM inoculation of the vaccine. Thus, the lower dose of RAdEa and RAdEs (7.5×10⁵ PFU) gave ˜60-fold higher antibody titers after the second booster when compared with the titers obtained with the vaccine. At the higher dose of 1×10⁸ PFU, RAdEs induced ˜250-fold higher titers than the vaccine on day 44.

Some investigators have carried out oral immunization of mice with RAds using Sodium bicarbonate buffer to neutralize the acid pH of the stomach (11, 40, 47). However, there are others who have carried out oral immunization of mice with RAds without the use of any buffer (3, 9, 42, 48). We have compared the immunogenicity in mice of RAdEs given orally (1×10⁸ PFU) with or without the bicarbonate buffer, FIG. 4 shows that no advantage was offered by the use of bicarbonate buffer during oral immunization with RAdEs. In fact, no effect of booster doses was seen when RAdEs was delivered orally using bicarbonate buffer and antibody titers on day 44 post-immunization were lower than in those mice that received the virus without bicarbonate buffer.

### EXAMPLE-12

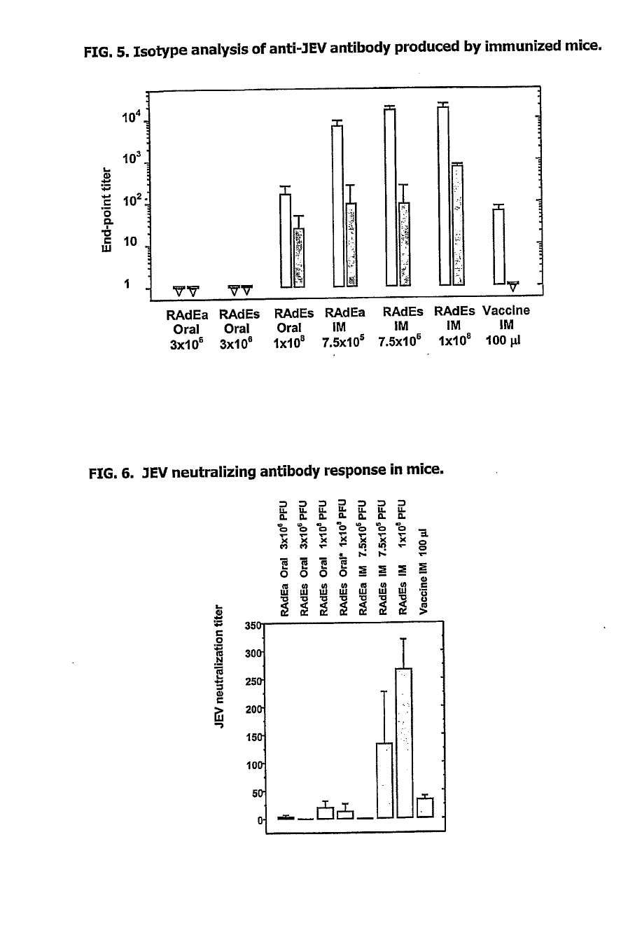

Isotype analyses of anti-JEV antibody produced by immunized mice: In order to analyze the quality of immune responses generated by the RAds, end-point titers of anti-JEV IgG1 and IgG2a antibodies were determined by ELISA. FIG. 5 shows that at lower dose of RAds given orally (3×10⁶ PFU) titers of anti-JEV IgG1 and IgG2a antibodies were below 25. When a higher dose of RAdEs (1×10⁸ PFU) was given orally, the majority of antibodies were of IgG1 type; the ratio of IgG1/IgG2a titers in this case was 6.48 indicating a Th2 type of immune response. When RAds were delivered through IM route, the anti-JEV antibody response was almost exclusively of IgG1 type. In case of mice immunized IM with RAdEa (7.5×10⁵ PFU), IgG1/IgG2a titer ratio was 68 and it was 64 in the case of RAdEs (7.5×10⁵ PFU)-immunized mice. At higher-dose of 1×10⁸ PFU, RAdEs again induced predominantly IgG1 type of anti-JEV antibodies; the ratio of IgG1/IgG2a titers in this case was 25. These results indicated that RAdEa and RAdEs induced an almost exclusive Th2 kind of immune response in mice when delivered IM. The IM immunization of mice with the vaccine also induced IgG1 dominated antibody response indicating a Th2 type immune response.

### EXAMPLE-13

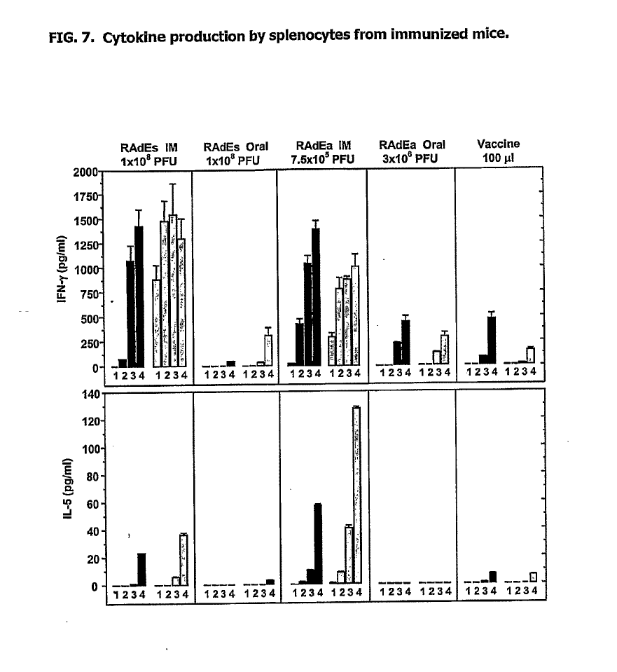

JEV neutralizing antibody response in mice immunized with Rads: Titers of JEV neutralizing antibodies were determined in serum samples obtained from the immunized mice at day 44 post-immunization. FIG. 6 shows that oral route of immunization induced very low JEV neutralizing antibodies. Similar to ELISA titers, the JEV neutralizing antibody titers were lower in mice immunized orally with RAdEs plus the bicarbonate buffer compared to titers in those mice immunized with RAdEs without the bicarbonate buffer, although the difference was statistically insignificant. Compared to RAdEa, RAdEs given IM induced higher JEV neutralizing titers and these were enhanced further when higher dose of virus was used for immunization. IM immunization with RAdEs induced significantly higher JEV neutralizing antibody titers than those induced by the vaccine.

### EXAMPLE-14

Cytokine secretion by splenocytes from immunized mice: Splenocytes prepared from the immunized mice were cultured in presence of JEV and synthesis of IFN-γ, IL-4 and IL-5 was studied on each day for the next 4 days. FIG. 7 shows that splenocytes from mice immunize with RAdEa or RAdEs by IM route secreted large amounts of IFN-γ. Splenocytes from mice immunized with the recombinant viruses through oral route made only small amounts of IFN-γ. Similarly, mice immunized with the vaccine made only small amounts of IFN-γ. Splenocytes from IM-immunized mice also made moderate amounts of IL-5 which was almost absent in the case of orally immunized mice or mice immunized with the vaccine. IL-4 was not detectable in any of the cases; the detection limit of IL-4 ELISA was 7.8 pg/ml. Similar pattern of cytokine secretion was observed when splenocytes were cultured in presence of JEV E protein

### EXAMPLE-15

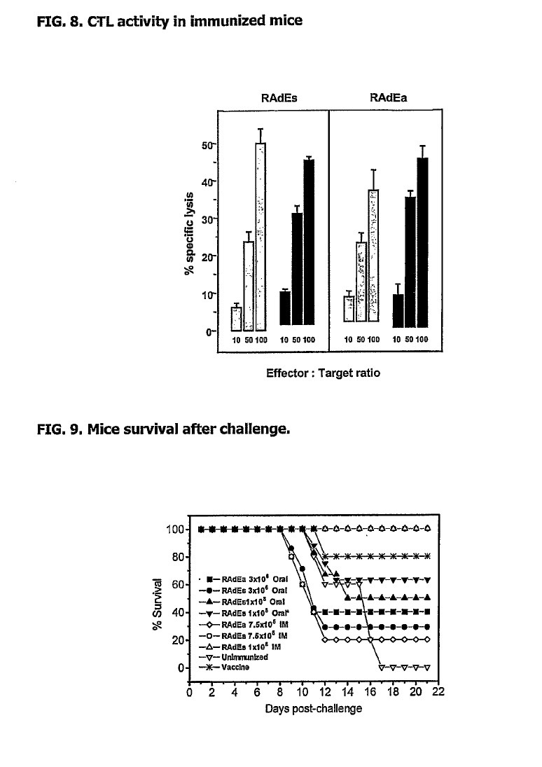

CTL activity in immunized mice: To study the generation of memory CTLs, splenocytes from immunized mice were stimulated in vitro with JEV and examined for cytotoxic activity against cells infected with JEV. FIG. 8 shows the results of CTL assays at various effector to target cell ratios. Thus, mice immunized with RAdEa or RAdEs through IM route showed significant CTL activity. No CTL activity was detectable in mice immunized with RAdEa or RAdEs through oral route. Similarly, unimmunized and Ad5-immunized mice or those immunized with the vaccine showed no CTL activity.

### EXAMPLE-16

Mice challenge studies: Mice immunized with RAds were challenged at day 44 post-immunization by intra-cerebral inoculation of a lethal dose (100 LD₅₀) of JEV. These mice were observed for mortality for 3 weeks after the challenge. All mice immunized with 7.5×10⁵ or 1×10⁸ PFU of RAdEs given IM survived the challenge while none of the unimmunized mice survived. Furthermore, none of the mice immunized IM or orally with 1×10⁸ PFU of E1/E3 Ad5 survived the challenge. About 50-60% protection was seen in mice immunized with 1×10⁸ PFU of RAdEs given orally. The level of protection was lower (30%) when mice were immunized orally with lower doses of RAdEs (3×10⁵ PFU). The level of protection afforded by RAdEa immunization was very low; about 40% mice survived from those immunized with 7.5×10⁵ PFU given IM and only about 20% mice survived from the group immunized with 3×10⁶ PFU given orally. In a separate experiment, 15 mice were immunized with 1×10⁸ PFU of RAdEs and given 2 booster doses on day 21 and 35. When challenged on day 44 post-immunization with 1000 LD₅₀ of JEV, given intra-cerebral, 100% of the immunized mice survived.

The flavivirus E protein has been the antigen of choice for vaccine development using modern methods of vaccinology such as the DNA vaccination or the use of recombinant virus for antigen delivery (10, 12, 16, 28-31). This has been so because E protein plays an important role in a number of processes, including viral attachment, membrane fusion and entry into the host cell (26). Besides, flavivirus E protein induces virus-neutralizing antibodies and CTLs (14, 14, 15, 22, 23, 34). It has been shown that protection against JEV is mainly antibody dependent, and virus-neutralizing antibodies alone are sufficient to impart protection (19, 32). This was also implied from our previous observation that the formalin-inactivated JEV vaccine, which did not induce CTLs, provided protection to vaccines against JEV (15). Thus, with a view to develop a recombinant virus-based JEV vaccine, we have constructed RAds synthesizing JEV E protein.

During replication of JEV, the E protein is expressed on the cell surface. Plasmid DNA vectors have been described that synthesize different forms of the E protein, such as the cytoplasmic, membrane-anchored or secretory (2, 6, 7, 15, 20). These different forms of JEV E protein were shown to induce different kind of immune responses. Similarly, recombinants of vaccinia expressing the secretory E protein of Japanese encephalitis or Dengue viruses were found to be highly immunogenic in mice (14, 27, 37). Previously, we had shown that truncated JEV E protein, where the membrane-anchor sequence had been removed by deletion of the C-terminal 102 amino acids, was actively secreted in the cell surroundings (15). We have now constructed RAds that synthesize the membrane-anchored or the secretory E protein.

Oral delivery of RAd has been shown to induce humoral and cellular immune responses to the protein encoded by the transgene, however, these responses have usually been weaker compared to those induced by the IM or intra-peritoneal delivery of RAd (21, 35, 42). In the present study too, oral immunization of mice by RAds resulted in significantly lower anti-JEV antibody titers when compared with the IM route of RAd delivery. Thus, mice immunized IM with 1×10⁸ PFU of RAdEs gave ˜60-fold higher anti-JEV antibody response than those immunized with the same dose of the virus given orally.

Use of Sodium carbonate buffer to neutralize the pH of the stomach made no perceptible difference to antibody titers when compared with the antibody titers induced by RAdEs given orally without the bicarbonate buffer. In fact, when bicarbonate buffer was used during oral immunization, the booster doses of RAdEs failed to enhance the anti-JEV antibody titers. For RAdEa too, oral delivery resulted in weaker anti-JEV antibody responses compared to the IM delivery of the recombinant. Thus, compared to oral delivery, IM inoculation of 7.5×10⁵ PFU of RAdEa (which was half the dose given orally) induced ˜100-fold higher anti-JEV antibody titers. The antibody titers were dose dependent. Thus higher doses of RAdEs (1×10⁸ PFU) delivered orally induced higher anti-JEV antibody titers. These titers were ˜4-fold higher than those induced by the commercial vaccine given IM. Importantly, IM inoculation of RAdEa or RAdEs at both the doses tested (7.5×10⁵ and 1×10⁸ PFU) resulted in significantly higher antibody responses than those given by the vaccine; 1×10⁸ PFU of RAdEs given IM induced ˜250-fold higher titer than the vaccine. Similar pattern was reflected in JEV neutralizing antibody titers when oral route of RAds delivery was compared with the IM route although the differences weren't so pronounced. Thus ˜20-fold higher JEV neutralizing antibody titers were induced by IM inoculation of 1×10⁸ PFU of RAdEs compared to oral delivery of the recombinant. Only statistically insignificant differences were noted in anti-JEV antibody titers induced by 7.5×10⁵ PFU of RAdEa and RAdEs given IM. However, there was significant difference in the JEV neutralizing antibody titers generated by the two RAds; a mean JEV neutralizing antibody titer of 10 was obtained when mice were immunized with RAdEa whereas it was 133 when mice were immunized with RAdEs. Higher JEV neutralizing antibody titers were recorded when higher doses of RAdEs were used for immunization. At both the doses tested (7.5×10⁵ and 1×10⁸ PFU) IM inoculation of RAdEs induced significantly higher JEV neutralizing antibody titers than the commercial vaccine. Thus a dose of 1×10⁸ PFU of RAdEs given IM induced 8-fold higher JEV neutralization titer than the vaccine. These differences in JEV neutralizing antibody titers induced by RAdEa and RAdEs by oral and IM inoculation were reflected in the level of protection afforded by these recombinants to the immunized mice against lethal JEV challenge. Thus both doses of RAdEs inducing JEV neutralizing antibody titers higher than the vaccine gave 100% protection. Immunization with RAdEs or RAdEa given orally gave only low levels of protection. Challenge experiments indicated that mice immunized IM with the adenovirus synthesizing the membrane-anchored form of JEV E protein did not develop protective immunity whereas those immunized with recombinant synthesizing the secretory form of JEV E protein developed robust anti-JEV protective immunity resulting in 100% protection. These results show that RAd-based JEV immunizations are superior to naked DNA immunizations, which imparted only about 50-60% protection in a mouse challenge model (15). It is interesting that level of protection was similar (50-60%) when mice were immunized with plasmid DNA synthesizing Ea or Es proteins (15) whereas in the present work Es induced significantly superior protective immune response than the Ea protein. This may be related to a more efficient delivery of JEV transgene using RAd than the direct injection of naked plasmid DNA for immunization. Our finding is, however, consistent with reports from others where truncated form of JEV or Dengue E protein (leading to its secretion) was found to be more immunogenic than the membrane-anchored form of the E protein (14, 37).

Poor anti-JEV antibody induction by oral immunization with RAds was also reflected in poor cytokine secretion by splenocytes in presence of JEV. While very little IFN-γ was secreted by splenocytes from mice immunized orally with RAdEs or RAdEa, significant amounts of IFN-γ were secreted by splenocytes obtained from mice immunized IM with RAdEa or RAdEs. Splenocytes from IM immunized mice also synthesized moderate amounts of IL-5 that was not detectable in cultures of splenocytes obtained from mice immunized orally with RAds. Mice immunized IM with both RAdEa and RAdEs had significant CTL activity, which was undetectable in mice immunized orally with RAds or IM with the vaccine. Oral immunization with RAdEs at lower dose resulted in IgG1 dominated immune responses; ratio of IgG1/IgG2a end-point titers was 6.5. This is consistent with studies on oral immunization of mice with RAd synthesizing rabies glycoprotein where abundance of anti rabies IgG1 was recorded (46). The IM inoculation of RAdEa and RAdEs also resulted in preponderance of IgG1 kind of antibodies. No data could be found in literature on antibody isotypes when adenovirus recombinants are delivered IM. Our results indicating the preponderance of IgG1 type antibodies, secretion of IFN-γ and IL-5 by splenocytes and induction of CTLs suggest that IM inoculation of mice with RAds synthesizing JEV E protein activates both the humoral and the cellular arms of the immune system, and immune responses of both Th1 and Th2 type are induced.

The results presented in this invention show that RAd synthesizing the secretory form of JEV E protein imparted robust immunity in mice against lethal dose of JEV given intra-cerebral. This makes RAdEs a potential candidate vaccine against JEV. Further, safety profile of the vaccine of the instant application was studied. The vaccine is found to be safe for administration. None of the immunized mice showed any obvious complications.

| Application Project | |
|---|---|
| ------------------- | |
| <120> | Title A recombinant vaccine against Japanese encephalitis virus |
| | (JEV) infection and a method thereof |

| <130> | AppFileReference: | IP 1340 |
|---|---|---|
| <140> | CurrentAppNumber: | 1516/Del/203 |
| <141> | CurrentFilingDate: | Dec. 04, 2003 |
| Sequence | | |
| -------- | | |
| <213> | OrganismName: | Artificial Sequence |
| <400> | PreSequenceString: | |

| | |
|---|---|
| cgtaactata acggtcctaa ggtagcgaaa gctcagatct ggatctcccg atcccctatg | 60 |
| gtcgactctc agtacaatct gctctgatgc cgcatagtta agccagtatc tgctccctgc | 120 |
| ttgtgtgttg gaggtcgctg agtagtgcgc gagcaaaatt taagctacaa caaggcaagg | 180 |
| cttgaccgac aattgcatga agaatctgct tagggttagg cgttttgcgc tgcttcgcga | 240 |
| tgtacgggcc agatatacgc gttgacattg attattgact agttattaat agtaatcaat | 300 |
| tacggggtca ttagttcata gcccatatat ggagttccgc gttacataac ttacggtaaa | 360 |
| tggcccgcct ggctgaccgc ccaacgaccc ccgcccattg acgtcaataa tgacgtatgt | 420 |
| tcccatagta acgccaatag ggactttcca ttgacgtcaa tgggtggact atttacggta | 480 |
| aactgcccac ttggcagtac atcaagtgta tcatatgcca atgaccttat ctattgacgt | 540 |
| caatgacggt aaatggcccg cctggcatta tgcccagtac atgaccttat gggactttcc | 600 |
| tacttggcag tacatctacg tattagtcat cgctattacc atggtgatgc ggttttggca | 660 |
| gtacatcaat gggcgtggat agcggtttga ctcacgggga tttccaagtc tccaccccat | 720 |
| tgacgtcaat gggagtttgt tttggcacca aaatcaacgg gactttccaa aatgtcgtaa | 780 |
| caactccgcc ccattgacgc aaatgggcgg taggcgtgta cggtgggagg tctatataag | 840 |
| cagagctctc tggctaacta gagaacccac tgcttactgg cttatcgaaa ttaatacgac | 900 |
| tcactatagg gagacccaag ctggctagcg tttaaacggg ccctctagac tcgagcggcc | 960 |
| gccactgtgc tggatgatcc gagctcggta ccatgtggct cgcaagcttg gcagttgtca | 1020 |
| tagcttgcgc aggagccatg aagttgtcga atttccaggg gaagcttttg atgaccatca | 1080 |
| acaacacgga cattgcagac gttatcgtga ttcccacctc aaaaggagag aacagatgct | 1140 |
| gggtccgggc aatcgacgtc ggttacatgt gtgaggacac catcacgtac gaatgtccta | 1200 |
| agcttactgc gggcaatgat ccagaggacg tggattgctg gtgtgacaac caagaagtct | 1260 |
| acgtccaata tggacggtgc acgcggacca ggcattccaa gcgaagcagg agatccgtgt | 1320 |
| cggtccagac acatggggag agttcactag tgaataaaaa agaggcttgg ctggattcaa | 1380 |
| cgaaagccac acgatatctc acgaaaactg agaactggat cataaggaat cctggctatg | 1440 |
| ctttcttggc ggcggtactt ggctggatgc ttggcagtaa caacggtcaa cgcgtggtat | 1500 |
| ttaccatcct cctgctgttg gtcgctccgg cttacagttt taattgtctg ggaatgggca | 1560 |
| atcgcgactt catagaagga gccagtggag ccacttgggt ggacttggtg ctagaaggag | 1620 |
| atagctgttt gacaatcatg gcaaacgata aaccaacatt ggatgtccgc atgattaaca | 1680 |
| tcgaagctag ccaacttgct gaggtcagaa gttattgcta tcatgcctca gtcactgaca | 1740 |
| tctcgacggt ggctcggtgc cccatgactg gagaagctca caacgagaag cgagctgata | 1800 |
| gtagctatgt gtgcaaacaa ggcttcactg atcgtgggtg gggcaacgga tgtggacttt | 1860 |
| tcgggaaggg aagcattgac acatgtgcaa aattctcctg caccagcaaa gcgattggga | 1920 |
| gaacaatcca gccagaaaac atcaaatacg aagttggcat ttttgtgcat ggaaccacca | 1980 |
| cttcggaaaa ccatgggaat tactcagcgc aagttggggc gtcccaggcg gcaaagttta | 2040 |
| ccgtaacacc caatgctcct tcgataaccc tcaaacttgg tgactacgga gaagtcacac | 2100 |
| tggactgtga gccaaggagt ggactgaaca ctgaagcgtt ttacgtcatg accgtggggt | 2160 |
| caaagtcatt tctggtccat agggaatggt ttcatgacct cgctctcccc tggacgtccc | 2220 |
| cttcgagcac agcgtggaga aacagagaac tcctcatgga atttgaagag gcgcacgcca | 2280 |
| caaaacagtc cgttgttgct cttggttcac aggaaggagg cctccatcag gcgttggcag | 2340 |
| gagccatcgt ggtggagtac tcaagctcag tgaagttaac atcaggccac ctgaaatgta | 2400 |
| ggctgaaaat ggacaaacta gctctgaaag gcacaaccta tggcatgtgt acagaaaaat | 2460 |
| tctcgttcgc gaaaaatccg gcggacactg gtcacggaac agttgtcatt gaactctcct | 2520 |
| actctgggag tgatggcccc tgcaaaattc cgattgtctc cgtcgcgagc ctcaatgaca | 2580 |
| tgactcctgt tgggcggctg gtgacagtga acccctttgt cgcggcttcc agtgccaact | 2640 |
| caaaggtgct ggtcgagatg gaacccccct tcggagactc ctatatcgtg gttggaaggg | 2700 |
| gagacaagca gatcaaccac cattggcaca aataataaag gatctgttgt ttgcccctcc | 2760 |
| cccgtgcctt ccttgaccct ggaaggtgcc actcccactg tcctttccta ataaaatgag | 2820 |
| gaaattgcat cgcattgtct gagtaggtgt cattctattc tggggggtgg ggtggggcag | 2880 |
| gacagcaagg gggaggattg ggaagacaat agcaggcatg ctggggatgc ggtgggctct | 2940 |
| atggcttctg aggcggaaag aaccagcaga tctgcagatc tgaattcatc tatgtcgggt | 3000 |
| gcggagaaag aggtaatgaa atggcatcga ctcgaagatc tgggcgtggt taagggtggg | 3060 |
| aaagaatata taaggtgggg gtcttatgta gttttgtatc tgttttgcag cagccgccgc | 3120 |
| cgccatgagc accaactcgt ttgatggaag cattgtgagc tcatatttga caacgcgcat | 3180 |
| gcccccatgg gccggggtgc gtcagaatgt gatgggctcc agcattgatg gtcgccccgt | 3240 |
| cctgcccgca aactctacta ccttgaccta cgagaccgtg tctggaacgc cgttggagac | 3300 |
| tgcagcctcc gccgccgctt cagccgctgc agccaccgcc cgcgggattg tgactgactt | 3360 |
| tgctttcctg agcccgcttg caagcagtgc agcttcccgt tcatccgccc gcgatgacaa | 3420 |
| gttgacggct cttttggcac aattggattc tttaacccgg gaacttaatg tcgtttctca | 3480 |
| gcagctgttg gatctgcgcc agcaggtttc tgccctgaag gcttcctccc ctcccaatgc | 3540 |
| ggtttaaaac ataaataaaa aaccagactc tgtttggatt tggatcaagc aagtgtcttg | 3600 |
| ctgtctttat ttaggggttt tgcgcgcgcg gtaggcccgg gaccagcggt ctcggtcgtt | 3660 |
| gagggtcctg tgtatttttt ccaggacgtg gtaaaggtga ctctggatgt tcagatacat | 3720 |
| gggcataagc ccgtctctgg ggtggaggta gcaccactgc agagcttcat gctgcggggt | 3780 |
| ggtgttgtag atgatccagt cgtagcagga gcgctgggcg tggtgcctaa aaatgtcttt | 3840 |
| cagtagcaag ctgattgcca ggggcaggcc cttggtgtaa gtgtttacaa agcggttaag | 3900 |
| ctgggatggg tgcatacgtg gggatatgag atgcatcttg gactgtattt ttaggttggc | 3960 |
| tatgttccca gccatatccc tccggggatt catgttgtgc agaaccacca gcacagtgta | 4020 |
| tccggtgcac ttgggaaatt tgtcatgtag cttagaagga aatgcgtgga agaacttgga | 4080 |
| gacgcccttg tgacctccaa gattttccat gcattcgtcc ataatgatgg caatgggccc | 4140 |
| acgggcggcg gcctgggcga agatatttct gggatcacta acgtcatagt tgtgttccag | 4200 |
| gatgagatcg tcataggcca tttttacaaa gcgcgggcgg agggtgccag actgcggtat | 4260 |
| aatggttcca tccggcccag gggcgtagtt accctcacag atttgcattt cccacgcttt | 4320 |
| gagttcagat ggggggatca tgtctacctg cggggcgatg aagaaaacgg tttccggggt | 4380 |
| aggggagatc agctgggaag aaagcaggtt cctgagcagc tgcgacttac cgcagccggt | 4440 |
| gggcccgtaa atcacaccta ttaccggctg caactggtag ttaagagagc tgcagctgcc | 4500 |
| gtcatccctg agcagggggg ccacttcgtt aagcatgtcc ctgactcgca tgttttccct | 4560 |
| gaccaaatcc gccagaaggc gctcgccgcc cagcgatagc agttcttgca aggaagcaaa | 4620 |
| gtttttcaac ggtttgagac cgtccgccgt aggcatgctt ttgagcgttt gaccaagcag | 4680 |
| ttccaggcgg tcccacagct cggtcacctg ctctacggca tctcgatcca gcatatctcc | 4740 |
| tcgtttcgcg ggttggggcg gctttcgctg tacggcagta gtcggtgctc gtccagacgg | 4800 |
| gccagggtca tgtctttcca cgggcgcagg gtcctcgtca gcgtagtctg ggtcacggtg | 4860 |
| aaggggtgcg ctccgggctg cgcgctggcc agggtgcgct tgaggctggt cctgctggtg | 4920 |
| ctgaagcgct gccggtcttc gccctgcgcg tcggccaggt agcatttgac catggtgtca | 4980 |
| tagtccagcc cctccgcggc gtggcccttg gcgcgcagct tgcccttgga ggaggcgccg | 5040 |
| cacgaggggc agtgcagact tttgagggcg tagagcttgg gcgcgagaaa taccgattcc | 5100 |
| ggggagtagg catccgcgcc gcaggccccg cagacggtct cgcattccac gagccaggtg | 5160 |
| agctctggcc gttcggggtc aaaaaccagg tttcccccat gctttttgat gcgtttctta | 5220 |
| cctctggttt ccatgagccg gtgtccacgc tcggtgacga aaaggctgtc cgtgtccccg | 5280 |
| tatacagact tgagaggcct gtcctcgagc ggtgttccgc ggtcctcctc gtatagaaac | 5340 |
| tcggaccact ctgagacaaa ggctcgcgtc caggccagca cgaaggaggc taagtgggag | 5400 |
| gggtagcggt cgttgtccac tagggggtcc actcgctcca gggtgtgaag acacatgtcg | 5460 |
| ccctcttcgg catcaaggaa ggtgattggt ttgtaggtgt aggccacgtg accgggtgtt | 5520 |
| cctgaagggg ggctataaaa gggggtgggg gcgcgttcgt cctcactctc ttccgcatcg | 5580 |
| ctgtctgcga gggccagctg ttggggtgag tactccctct gaaaagcggg catgacttct | 5640 |
| gcgctaagat tgtcagtttc caaaaacgag gaggatttga tattcacctg gcccgcggtg | 5700 |
| atgcctttga gggtggccgc atccatctgg tcagaaaaga caatcttttt gttgtcaagc | 5760 |
| ttggtggcaa acgacccgta gagggcgttg gacagcaact tggcgatgga gcgcagggtt | 5820 |
| tggtttttgt cgcgatcggc gcgctccttg gccgcgatgt ttagctgcac gtattcgcgc | 5880 |
| gcaacgcacc gccattcggg aaagacggtg gtgcgctcgt cgggcaccag gtgcacgcgc | 5940 |
| caaccgcggt tgtgcagggt gacaaggtca acgctggtgg ctacctctcc gcgtaggcgc | 6000 |
| tcgttggtcc agcagaggcg gccgcccttg cgcgagcaga atggcggtag ggggtctagc | 6060 |
| tgcgtctcgt ccggggggtc tgcgtccacg gtaaagaccc cgggcagcag gcgcgcgtcg | 6120 |
| aagtagtcta tcttgcatcc ttgcaagtct agcgcctgct gccatgcgcg ggcggcaagc | 6180 |
| gcgcgctcgt atgggttgag tgggggaccc catggcatgg ggtgggtgag cgcggaggcg | 6240 |
| tacatgccgc aaatgtcgta aacgtagagg ggctctctga gtattccaag atatgtaggg | 6300 |
| tagcatcttc caccgcggat gctggcgcgc acgtaatcgt atagttcgtg cgagggagcg | 6360 |
| aggaggtcgg gaccgaggtt gctacgggcg ggctgctctg ctcggaagac tatctgcctg | 6420 |
| aagatggcat gtgagttgga tgatatggtt ggacgctgga agacgttgaa gctggcgtct | 6480 |
| gtgagaccta ccgcgtcacg cacgaaggag gcgtaggagt cgcgcagctt gttgaccagc | 6540 |
| tcggcggtga cctgcacgtc tagggcgcag tagtccaggg tttccttgat gatgtcatac | 6600 |
| ttatcctgtc cctttttttt ccacagctcg cggttgagga caaactcttc gcggtctttc | 6660 |
| cagtactctt ggatcggaaa cccgtcggcc tccgaacggt aagagcctag catgtagaac | 6720 |
| tggttgacgg cctggtaggc gcagcatccc ttttctacgg gtagcgcgta tgcctgcgcg | 6780 |
| gccttccgga gcgaggtgtg ggtgagcgca aaggtgtccc tgaccatgac tttgaggtac | 6840 |
| tggtatttga agtcagtgtc gtcgcatccg ccctgctccc agagcaaaaa gtccgtgcgc | 6900 |
| tttttggaac gcggatttgg cagggcgaag gtgacatcgt tgaagagtat ctttcccgcg | 6960 |
| cgaggcataa agttgcgtgt gatgcggaag ggtcccggca cctcggaacg gttgttaatt | 7020 |
| acctgggcgg cgagcacgat ctcgtcaaag ccgttgatgt tgtggcccac aatgtaaagt | 7080 |
| tccaagaagc gcgggatgcc cttgatggaa ggcaattttt taagttcctc gtaggtgagc | 7140 |
| tcttcagggg agctgagccc gtgctctgaa agggcccaat ctgcaagatg agggttggaa | 7200 |
| gcgacgaatg agctccacag gtcacgggcc attagcattt gcaggtggtc gcgaaaggtc | 7260 |
| ctaaactggc gacctatggc cattttttct ggggtgatgc agtagaaggt aagcgggtct | 7320 |
| tgttcccagc ggtcccatcc aaggttcgcg gctaggtctc gcgcggcagt cactagaggc | 7380 |
| tcatctccgc cgaacttcat gaccagcatg aagggcacga gctgcttccc aaaggccccc | 7440 |
| atccaagtat aggtctctac atcgtaggtg acaaagagac gctcggtgcg aggatgcgag | 7500 |
| ccgatcggga agaactggat ctcccgccac caattggagg agtggctatt gatgtggtga | 7560 |
| aagtagaagt ccctgcgacg ggccgaacac tcgtgctggc ttttgtaaaa acgtgcgcag | 7620 |
| tactggcagc ggtgcacggg ctgtacatcc tgcacgaggt tgacctgacg accgcgcaca | 7680 |
| aggaagcaga gtgggaattt gagcccctcg cctggcgggt ttggctggtg gtcttctact | 7740 |
| tcggctgctt gtccttgacc gtctggctgc tcgaggggag ttacggtgga tcggaccacc | 7800 |
| acgccgcgcg agcccaaagt ccagatgtcc gcgcgcggcg gtcggagctt gatgacaaca | 7860 |
| tcgcgcagat gggagctgtc catggtctgg agctcccgcg gcgtcaggtc aggcgggagc | 7920 |
| tcctgcaggt ttacctcgca tagacgggtc agggcgcggg ctagatccag gtgataccta | 7980 |
| atttccaggg gctggttggt ggcggcgtcg atggcttgca agaggccgca tccccgcggc | 8040 |
| gcgactacgg taccgcgcgg cgggcggtgg gccgcggggg tgtccttgga tgatgcatct | 8100 |
| aaaagcggtg acgcgggcga gcccccggag ataggggggg ctccggaccc gccgggagag | 8160 |
| ggggcagggg cacgtcggcg ccgcgcgcgg gcaggagctg gtgctgcgcg cgtaggttgc | 8220 |
| tggcgaacgc gacgacgcgg cggttgatct cctgaatctg gcgcctctgc gtgaagacga | 8280 |
| cgggcccggt gagcttgaac ctgaaagaga gttcgacaga atcaatttcg gtgtcgttga | 8340 |
| cggcggcctg gcgcaaaatc tcctgcacgt ctcctgagtt gtcttgatag gcgatctcgg | 8400 |
| ccatgaactg ctcgatctct tcctcctgga gatctccgcg tccggctcgc tccacggtgg | 8460 |
| cggcgaggtc gttggaaatg cgggccatga gctgcgagaa ggcgttgagg cctccctcgt | 8520 |
| tccagacgcg gctgtagacc acgccccctt cggcatcgcg ggcgcgcatg accacctgcg | 8580 |
| cgagattgag ctccacgtgc cgggcgaaga cggcgtagtt tcgcaggcgc tgaaagaggt | 8640 |
| agttgagggt ggtggcggtg tgttctgcca cgaagaagta cataacccag cgtcgcaacg | 8700 |
| tggattcgtt gatatccccc aaggcctcaa ggcgctccat ggcctcgtag aagtccacgg | 8760 |
| cgaagttgaa aaactgggag ttgcgcgccg acacggttaa ctcctcctcc agaagacgga | 8820 |
| tgagctcggc gacagtgtcg cgcacctcgc gctcaaaggc tacaggggcc tcttcttctt | 8880 |
| cttcaatctc ctcttccata agggcctccc cttcttcttc ttctggcggc ggtgggggag | 8940 |
| gggggacacg gcggcgacga cggcgcaccg ggaggcggtc gacaaagcgc tcgatcatct | 9000 |
| ccccgcggcg acggcgcatg gtctcggtga cggcgcggcc gttctcgcgg gggcgcagtt | 9060 |
| ggaagacgcc gcccgtcatg tcccggttat gggttggcgg ggggctgcca tgcggcaggg | 9120 |
| atacggcgct aacgatgcat ctcaacaatt gttgtgtagg tactccgccg ccgagggacc | 9180 |
| tgagcgagtc cgcatcgacc ggatcggaaa acctctcgag aaaggcgtct aaccagtcac | 9240 |
| agtcgcaagg taggctgagc accgtggcgg gcggcagcgg gcggcggtcg gggttgtttc | 9300 |
| tggcggaggt gctgctgatg acgtaattaa agtaggcggt cttgagacgg cggatggtcg | 9360 |
| acagaagcac catgtccttg ggtccggcct gctgaatgcg caggcggtcg gccatgcccc | 9420 |
| aggcttcgtt ttgacatcgg cgcaggtctt tgtagtagtc ttgcatgagc ctttctaccg | 9480 |
| gcacttcttc ttctccttcc tcttgtcctg catctcttgc atctatcgct gcggcggcgg | 9540 |
| cggagtttgg ccgtaggtgg cgccctcttc ctcccatgcg tgtgaccccg aagcccctca | 9600 |
| tcggctgaag cagggctagg tcggcgacaa cgcgctcggc taatatggcc tgctgcacct | 9660 |
| gcgtgagggt agactggaag tcatccatgt ccacaaagcg gtggtatgcg cccgtgttga | 9720 |
| tggtgtaagt gcagttggcc ataacggacc agttaacggt ctggtgaccc ggctgcgaga | 9780 |
| gctcggtgta cctgagacgc gagtaagccc tcgagtcaaa tacgtagtcg ttgcaagtcc | 9840 |
| gcaccaggta ctggtatccc accaaaaagt gcggcggcgg ctggcggtag aggggccagc | 9900 |
| gtagggtggc cggggctccg ggggcgagat cttccaacat aaggcgatga tatccgtaga | 9960 |
| tgtacctgga catccaggtg atgccggcgg cggtggtgga ggcgcgcgga aagtcgcgga | 10020 |
| cgcggttcca gatgttgcgc agcggcaaaa agtgctccat ggtcgggacg ctctggccgg | 10080 |
| tcaggcgcgc gcaatcgttg acgctctagc gtgcaaaagg agagcctgta agcgggcact | 10140 |
| cttccgtggt ctggtggata aattcgcaag ggtatcatgg cggacgaccg gggttcgagc | 10200 |
| cccgtatccg gccgtccgcc gtgatccatg cggttaccgc ccgcgtgtcg aacccaggtg | 10260 |
| tgcgacgtca gacaacgggg gagtgctcct tttggcttcc ttccaggcgc ggcggctgct | 10320 |
| gcgctagctt ttttggccac tggccgcgcg cagcgtaagc ggttaggctg gaaagcgaaa | 10380 |
| gcattaagtg gctcgctccc tgtagccgga gggttatttt ccaagggttg agtcgcggga | 10440 |
| cccccggttc gagtctcgga ccggccggac tgcggcgaac gggggtttgc ctccccgtca | 10500 |
| tgcaagaccc cgcttgcaaa ttcctccgga aacagggacg agcccctttt ttgcttttcc | 10560 |
| cagatgcatc cggtgctgcg gcagatgcgc ccccctcctc agcagcggca agagcaagag | 10620 |
| cagcggcaga catgcagggc accctcccct cctcctaccg cgtcaggagg ggcgacatcc | 10680 |
| gcggttgacg cggcagcaga tggtgattac gaacccccgc ggcgccgggc ccggcactac | 10740 |
| ctggacttgg aggagggcga gggcctggcg cggctaggag cgccctctcc tgagcggcac | 10800 |
| ccaagggtgc agctgaagcg tgatacgcgt gaggcgtacg tgccgcggca gaacctgttt | 10860 |
| cgcgaccgcg agggagagga gcccgaggag atgcgggatc gaaagttcca cgcagggcgc | 10920 |
| gagctgcggc atggcctgaa tcgcgagcgg ttgctgcgcg aggaggactt tgagcccgac | 10980 |
| gcgcgaaccg ggattagtcc cgcgcgcgca cacgtggcgg ccgccgacct ggtaaccgca | 11040 |
| tacgagcaga cggtgaacca ggagattaac tttcaaaaaa gctttaacaa ccacgtgcgt | 11100 |
| acgcttgtgg cgcgcgagga ggtggctata ggactgatgc atctgtggga ctttgtaagc | 11160 |
| gcgctggagc aaaacccaaa tagcaagccg ctcatggcgc agctgttcct tatagtgcag | 11220 |
| cacagcaggg acaacgaggc attcagggat gcgctgctaa acatagtaga gcccgagggc | 11280 |
| cgctggctgc tcgatttgat aaacatcctg cagagcatag tggtgcagga gcgcagcttg | 11340 |
| agcctggctg acaaggtggc cgccatcaac tattccatgc ttagcctggg caagttttac | 11400 |
| gcccgcaaga tataccatac cccttacgtt cccatagaca aggaggtaaa gatcgagggg | 11460 |
| ttctacatgc gcatggcgct gaaggtgctt accttgagcg acgacctggg cgtttatcgc | 11520 |
| aacgagcgca tccacaaggc cgtgagcgtg agccggcggc gcgagctcag cgaccgcgag | 11580 |
| ctgatgcaca gcctgcaaag ggccctggct ggcacgggca gcggcgatag agaggccgag | 11640 |
| tcctactttg acgcgggcgc tgacctgcgc tgggccccaa gccgacgcgc cctggaggca | 11700 |
| gctggggccg gacctgggct ggcggtggca cccgcgcgcg ctggcaacgt cggcggcgtg | 11760 |
| gaggaatatg acgaggacga tgagtacgag ccagaggacg gcgagtacta agcggtgatg | 11820 |
| tttctgatca gatgatgcaa gacgcaacgg acccggcggt gcgggcggcg ctgcagagcc | 11880 |
| agccgtccgg ccttaactcc acggacgact ggcgccaggt catggaccgc atcatgtcgc | 11940 |
| tgactgcgcg caatcctgac gcgttccggc agcagccgca ggccaaccgg ctctccgcaa | 12000 |
| ttctggaagc ggtggtcccg gcgcgcgcaa accccacgca cgagaaggtg ctggcgatcg | 12060 |
| taaacgcgct ggccgaaaac agggccatcc ggcccgacga ggccggcctg gtctacgacg | 12120 |
| cgctgcttca gcgcgtggct cgttacaaca gcggcaacgt gcagaccaac ctggaccggc | 12180 |
| tggtggggga tgtgcgcgag gccgtggcgc agcgtgagcg cgcgcagcag cagggcaacc | 12240 |
| tgggctccat ggttgcacta aacgccttcc tgagtacaca gcccgccaac gtgccgcggg | 12300 |
| gacaggagga ctacaccaac tttgtgagcg cactgcggct aatggtgact gagacaccgc | 12360 |
| aaagtgaggt gtaccagtct gggccagact attttttcca gaccagtaga caaggcctgc | 12420 |
| agaccgtaaa cctgagccag gctttcaaaa acttgcaggg gctgtggggg gtgcgggctc | 12480 |
| ccacaggcga ccgcgcgacc gtgtctagct tgctgacgcc caactcgcgc ctgttgctgc | 12540 |
| tgctaatagc gcccttcacg gacagtggca gcgtgtcccg ggacacatac ctaggtcact | 12600 |
| tgctgacact gtaccgcgag gccataggtc aggcgcatgt ggacgagcat actttccagg | 12660 |
| agattacaag tgtcagccgc gcgctggggc aggaggacac gggcagcctg gaggcaaccc | 12720 |
| taaactacct gctgaccaac cggcggcaga agatcccctc gttgcacagt ttaaacagcg | 12780 |
| aggaggagcg cattttgcgc tacgtgcagc agagcgtgag ccttaacctg atgcgcgacg | 12840 |
| gggtaacgcc cagcgtggcg ctggacatga ccgcgcgcaa catggaaccg ggcatgtatg | 12900 |
| cctcaaaccg gccgtttatc aaccgcctaa tggactactt gcatcgcgcg gccgccgtga | 12960 |
| accccgagta tttcaccaat gccatcttga acccgcactg gctaccgccc cctggtttct | 13020 |
| acaccggggg attcgaggtg cccgagggta acgatggatt cctctgggac gacatagacg | 13080 |
| acagcgtgtt ttccccgcaa ccgcagaccc tgctagagtt gcaacagcgc gagcaggcag | 13140 |
| aggcggcgct gcgaaaggaa agcttccgca ggccaagcag cttgtccgat ctaggcgctg | 13200 |
| cggccccgcg gtcagatgct agtagcccat ttccaagctt gatagggtct cttaccagca | 13260 |
| ctcgcaccac ccgcccgcgc ctgctgggcg aggaggagta cctaaacaac tcgctgctgc | 13320 |
| agccgcagcg cgaaaaaaac ctgcctccgg catttcccaa caacgggata gagagcctag | 13380 |
| tggacaagat gagtagatgg aagacgtacg cgcaggagca cagggacgtg ccaggcccgc | 13440 |
| gcccgcccac ccgtcgtcaa aggcacgacc gtcagcgggg tctggtgtgg gaggacgatg | 13500 |
| actcggcaga cgacagcagc gtcctggatt tgggagggag tggcaacccg tttgcgcacc | 13560 |
| ttcgccccag gctggggaga atgttttaaa aaaaaaaaaa gcatgatgca aaataaaaaa | 13620 |
| ctcaccaagg ccatggcacc gagcgttggt tttcttgtat tccccttagt atgcggcgcg | 13680 |
| cggcgatgta tgaggaaggt cctcctccct cctacgagag tgtggtgagc gcggcgccag | 13740 |
| tggcggcggc gctgggttct cccttcgatg ctcccctgga cccgccgttt gtgcctccgc | 13800 |
| ggtacctgcg gcctaccggg gggagaaaca gcatccgtta ctctgagttg gcacccctat | 13860 |
| tcgacaccac ccgtgtgtac ctggtggaca acaagtcaac ggatgtggca tccctgaact | 13920 |
| accagaacga ccacagcaac tttctgacca cggtcattca aaacaatgac tacagcccgg | 13980 |
| gggaggcaag cacacagacc atcaatcttg acgaccggtc gcactggggc ggcgacctga | 14040 |
| aaaccatcct gcataccaac atgccaaatg tgaacgagtt catgtttacc aataagttta | 14100 |
| aggcgcgggt gatggtgtcg cgcttgccta ctaaggacaa tcaggtggag ctgaaatacg | 14160 |
| agtgggtgga gttcacgctg cccgagggca actactccga gaccatgacc atagacctta | 14220 |
| tgaacaacgc gatcgtggag cactacttga aagtgggcag acagaacggg gttctggaaa | 14280 |
| gcgacatcgg ggtaaagttt gacacccgca acttcagact ggggtttgac cccgtcactg | 14340 |
| gtcttgtcat gcctggggta tatacaaacg aagccttcca tccagacatc attttgctgc | 14400 |
| caggatgcgg ggtggacttc acccacagcc gcctgagcaa cttgttgggc atccgcaagc | 14460 |
| ggcaaccctt ccaggagggc tttaggatca cctacgatga tctggagggt ggtaacattc | 14520 |
| ccgcactgtt ggatgtggac gcctaccagg cgagcttgaa agatgacacc gaacagggcg | 14580 |
| ggggtggcgc aggcggcagc aacagcagtg gcagcggcgc ggaagagaac tccaacgcgg | 14640 |
| cagccgcggc aatgcagccg gtggaggaca tgaacgatca tgccattcgc ggcgacacct | 14700 |
| ttgccacacg ggctgaggag aagcgcgctg aggccgaagc agcggccgaa gctgccgccc | 14760 |
| ccgctgcgca acccgaggtc gagaagcctc agaagaaacc ggtgatcaaa cccctgacag | 14820 |
| aggacagcaa gaaacgcagt tacaacctaa taagcaatga cagcaccttc acccagtacc | 14880 |
| gcagctggta ccttgcatac aactacggcg accctcagac cggaatccgc tcatggaccc | 14940 |
| tgctttgcac tcctgacgta acctgcggct cggagcaggt ctactggtcg ttgccagaca | 15000 |
| tgatgcaaga ccccgtgacc ttccgctcca cgcgccagat cagcaacttt ccggtggtgg | 15060 |
| gcgccgagct gttgcccgtg cactccaaga gcttctacaa cgaccaggcc gtctactccc | 15120 |
| aactcatccg ccagtttacc tctctgaccc acgtgttcaa tcgctttccc gagaaccaga | 15180 |
| ttttggcgcg cccgccagcc cccaccatca ccaccgtcag tgaaaacgtt cctgctctca | 15240 |
| cagatcacgg gacgctaccg ctgcgcaaca gcatcggagg agtccagcga gtgaccatta | 15300 |
| ctgacgccag acgccgcacc tgcccctacg tttacaaggc cctgggcata gtctcgccgc | 15360 |
| gcgtcctatc gagccgcact ttttgagcaa gcatgtccat ccttatatcg cccagcaata | 15420 |
| acacaggctg gggcctgcgc ttcccaagca agatgtttgg cggggccaag aagcgctccg | 15480 |
| accaacaccc agtgcgcgtg cgcgggcact accgcgcgcc ctggggcgcg caccaacgcg | 15540 |
| gccgcactgg gcgcaccacc gtcgatgacg ccatcgacgc ggtggtggag gaggcgcgca | 15600 |
| actacacgcc cacgccgcca ccagtgtcca cagtggacgc ggccattcag accgtggtgc | 15660 |
| gcggagcccg gcgctatgct aaaatgaaga gacggcggag gcgcgtagca cgtcgccacc | 15720 |
| gccgccgacc cggcactgcc gcccaacgcg cggcggcggc cctgcttaac cgcgcacgtc | 15780 |
| gcaccggccg acgggcggcc atgcgggccg ctcgaaggct ggccgcgggt attgtcactg | 15840 |
| tgccccccag gtccaggcga cgagcggccg ccgcagcagc cgcggccatt agtgctatga | 15900 |
| ctcagggtcg caggggcaac gtgtattggg tgcgcgactc ggttagcggc ctgcgcgtgc | 15960 |
| ccgtgcgcac ccgccccccg cgcaactaga ttgcaagaaa aaactactta gactcgtact | 16020 |
| gttgtatgta tccagcggcg gcggcgcgca acgaagctat gtccaagcgc aaaatcaaag | 16080 |
| aagagatgct ccaggtcatc gcgccggaga tctatggccc cccgaagaag gaagagcagg | 16140 |
| attacaagcc ccgaaagcta aagcgggtca aaaagaaaaa gaaagatgat gatgatgaac | 16200 |
| ttgacgacga ggtggaactg ctgcacgtta ccgcgcccag gcgacgggta cagtggaaag | 16260 |
| gtcgacgcgt aaaacgtgtt ttgcgacccg gcaccaccgt agtctttacg cccggtgagc | 16320 |
| gctccacccg cacctacaag cgcgtgtatg atgaggtgta cggcgacgag gacctgcttg | 16380 |
| agcaggccaa cgagcgcctc ggggagtttg cctacggaaa gcggcataag gacatgctgg | 16440 |
| cgttgccgct ggacgagggc aacccaacac ctagcctaaa gcccgtaaca ctgcagcagg | 16500 |
| tgctgcccgc gcttgcaccg tccgaagaaa agcgcggcct aaagcgcgag tctggtgact | 16560 |
| tggcacccac cgtgcagctg atggtaccca agcgccagcg actggaagat gtcttggaaa | 16620 |
| aaatgaccgt ggaacctggg ctggagcccg aggtccgcgt gcggccaatc aagcaggtgg | 16680 |
| cgccgggact gggcgtgcag accgtggacg ttcagatacc cactaccagt agcaccagta | 16740 |
| ttgccaccgc cacagagggc atggagacac aaacgtcccc ggttgcctca gcggtggcgg | 16800 |
| atgccgcggt gcaggcggtc gctgcggccg cgtccaagac ctctacggag gtgcaaacgg | 16860 |
| acccgtggat gtttcgcgtt tcagcccccc ggcgcccgcg ccgttcgagg aagtacggcg | 16920 |
| ccgccagcgc gctactgccc gaatatgccc tacatccttc cattgcgcct acccccggct | 16980 |
| atcgtggcta cacctaccgc cccagaagac gagcaactac ccgacgccga accaccactg | 17040 |
| gaacccgccg ccgccgtcgc cgccgccagc ccgtgctggc cccgatttcc gtgcgcaggg | 17100 |
| tggctcgcga aggaggcagg accctggtgc tgccaacagc gcgccaccac cccagcatcg | 17160 |
| tttaaaagcc ggtctttgtg gttcttgcag atatggccct cacctgccgc ctccgtttcc | 17220 |
| cggtgccggg attccgagga agaatgcacc gtaggagggg catggccggc cacggcctga | 17280 |
| cgggcggcat gcgtcgtgcg caccaccggc ggcggcgcgc gtcgcaccgt cgcatgcgcg | 17340 |
| gcggtatcct gcccctcctt attccactga tcgccgcggc gattggcgcc gtgcccggaa | 17400 |
| ttgcatccgt ggccttgcag gcgcagagac actgattaaa aacaagttgc atgtggaaaa | 17460 |
| atcaaaataa aaagtctgga ctctcacgct cgcttggtcc tgtaactatt ttgtagaatg | 17520 |
| gaagacatca actttgcgtc tctggccccg cgacacggct cgcgcccgtt catgggaaac | 17580 |
| tggcaagata tcggcaccag caatatgagc ggtggcgcct tcagctgggg ctcgctgtgg | 17640 |
| agcggcatta aaaatttcgg ttccaccgtt aagaactatg gcagcaaggc ctggaacagc | 17700 |
| agcacaggcc agatgctgag ggataagttg aaagagcaaa atttccaaca aaaggtggta | 17760 |
| gatggcctgg cctctggcat tagcggggtg gtggacctgg ccaaccaggc agtgcaaaat | 17820 |
| aagattaaca gtaagcttga tccccgccct cccgtagagg agcctccacc ggccgtggag | 17880 |
| acagtatctc cagaggggcg tggcgaaaag cgtccgcgcc ccgacaggga agaaactctg | 17940 |
| gtgacgcaaa tagacgagcc cccctcgtac gaggaggcac taaagcaagg cctgcccacc | 18000 |
| acccgtccca tcgcgcccat ggctaccgga gtgctgggcc agcacacacc cgtaacgctg | 18060 |
| gacctgcctc cccccgccga cacccagcag aaacctgtgc tgccaggccc gaccgccgtt | 18120 |
| gttgtaaccc gtcctagccg cgcgtccctg cgccgcgccg ccagcggtcc gcgatcgttg | 18180 |
| cggcccgtag ccagtggcaa ctggcaaagc acactgaaca gcatcgtggg tctgggggtg | 18240 |
| caatccctga agcgccgacg atgcttctga tagctaacgt gtcgtatgtg tgtcatgtat | 18300 |
| gcgtccatgt cgccgccaga ggagctgctg agccgccgcg cgcccgcttt ccaagatggc | 18360 |
| taccccttcg atgatgccgc agtggtctta catgcacatc tcgggccagg acgcctcgga | 18420 |
| gtacctgagc cccgggctgg tgcagtttgc ccgcgccacc gagacgtact tcagcctgaa | 18480 |
| taacaagttt agaaacccca cggtggcgcc tacgcacgac gtgaccacag accggtccca | 18540 |
| gcgtttgacg ctgcggttca tccctgtgga ccgtgaggat actgcgtact cgtacaaggc | 18600 |
| gcggttcacc ctagctgtgg gtgataaccg tgtgctggac atggcttcca cgtactttga | 18660 |
| catccgcggc gtgctggaca ggggccctac ttttaagccc tactctggca ctgcctacaa | 18720 |
| cgccctggct cccaagggtg ccccaaatcc ttgcgaatgg gatgaagctg ctactgctct | 18780 |
| tgaaataaac ctagaagaag aggacgatga caacgaagac gaagtagacg agcaagctga | 18840 |
| gcagcaaaaa actcacgtat ttgggcaggc gccttattct ggtataaata ttacaaagga | 18900 |
| gggtattcaa ataggtgtcg aaggtcaaac acctaaatat gccgataaaa catttcaacc | 18960 |
| tgaacctcaa ataggagaat ctcagtggta cgaaacagaa attaatcatg cagctgggag | 19020 |
| agtcctaaaa aagactaccc caatgaaacc atgttacggt tcatatgcaa aacccacaaa | 19080 |
| tgaaaatgga gggcaaggca ttcttgtaaa gcaacaaaat ggaaagctag aaagtcaagt | 19140 |
| ggaaatgcaa tttttctcaa ctactgaggc agccgcaggc aatggtgata acttgactcc | 19200 |
| taaagtggta ttgtacagtg aagatgtaga tatagaaacc ccagacactc atatttctta | 19260 |
| catgcccact attaaggaag gtaactcacg agaactaatg ggccaacaat ctatgcccaa | 19320 |
| caggcctaat tacattgctt ttagggacaa ttttattggt ctaatgtatt acaacagcac | 19380 |
| gggtaatatg ggtgttctgg cgggccaagc atcgcagttg aatgctgttg tagatttgca | 19440 |
| agacagaaac acagagcttt cataccagct tttgcttgat tccattggtg atagaaccag | 19500 |
| gtacttttct atgtggaatc aggctgttga cagctatgat ccagatgtta gaattattga | 19560 |
| aaatcatgga actgaagatg aacttccaaa ttactgcttt ccactgggag gtgtgattaa | 19620 |
| tacagagact cttaccaagg taaaacctaa aacaggtcag gaaaatggat gggaaaaaga | 19680 |
| tgctacagaa ttttcagata aaaatgaaat aagagttgga aataattttg ccatggaaat | 19740 |
| caatctaaat gccaacctgt ggagaaattt cctgtactcc aacatagcgc tgtatttgcc | 19800 |
| cgacaagcta aagtacagtc cttccaacgt aaaaatttct gataacccaa acacctacga | 19860 |
| ctacatgaac aagcgagtgg tggctcccgg gctagtggac tgctacatta accttggagc | 19920 |
| acgctggtcc cttgactata tggacaacgt caacccattt aaccaccacc gcaatgctgg | 19980 |
| cctgcgctac cgctcaatgt tgctgggcaa tggtcgctat gtgcccttcc acatccaggt | 20040 |
| gcctcagaag ttctttgcca ttaaaaacct ccttctcctg ccgggctcac acacctacga | 20100 |
| gtggaacttc aggaaggatg ttaacatggt tctgcagagc tccctaggaa atgacctaag | 20160 |
| ggttgacgga gccagcatta agtttgatag catttgcctt tacgccacct tcttccccat | 20220 |
| ggcccacaac accgcctcca cgcttgaggc catgcttaga aacgacacca acgaccagtc | 20280 |
| ctttaacgac tatctctccg ccgccaacat gctctaccct atacccgcca acgctaccaa | 20340 |
| cgtgcccata tccatcccct cccgcaactg ggcggctttc cgcggctggg ccttcacgcg | 20400 |
| ccttaagact aaggaaaccc catcactggg ctcgggctac gacccttatt acacctactc | 20460 |
| tggctctata ccctacctag atggaacctt ttacctcaac cacaccttta agaaggtggc | 20520 |
| cattaccttt gactcttctg tcagctggcc tggcaatgac cgcctgctta cccccaacga | 20580 |
| gtttgaaatt aagcgctcag ttgacgggga gggttacaac gttgcccagt gtaacatgac | 20640 |
| caaagactgg ttcctggtac aaatgctagc taactataac attggctacc agggcttcta | 20700 |
| tatcccagag agctacaagg accgcatgta ctccttcttt agaaacttcc agcccatgag | 20760 |
| ccgtcaggtg gtggatgata ctaaatacaa ggactaccaa caggtgggca tcctacacca | 20820 |
| acacaacaac tctggatttg ttggctacct tgcccccacc atgcgcgaag gacaggccta | 20880 |
| ccctgctaac ttcccctatc cgcttatagg caagaccgca gttgacagca ttacccagaa | 20940 |
| aaagtttctt tgcgatcgca ccctttggcg catcccattc tccagtaact ttatgtccat | 21000 |
| gggcgcactc acagacctgg gccaaaacct tctctacgcc aactccgccc acgcgctaga | 21060 |
| catgactttt gaggtggatc ccatggacga gcccaccctt ctttatgttt tgtttgaagt | 21120 |
| ctttgacgtg gtccgtgtgc accagccgca ccgcggcgtc atcgaaaccg tgtacctgcg | 21180 |
| cacgcccttc tcggccggca acgccacaac ataaagaagc aagcaacatc aacaacagct | 21240 |
| gccgccatgg gctccagtga gcaggaactg aaagccattg tcaaagatct tggttgtggg | 21300 |
| ccatattttt tgggcaccta tgacaagcgc tttccaggct ttgtttctcc acacaagctc | 21360 |
| gcctgcgcca tagtcaatac ggccggtcgc gagactgggg gcgtacactg gatggccttt | 21420 |
| gcctggaacc cgcactcaaa aacatgctac ctctttgagc cctttggctt ttctgaccag | 21480 |
| cgactcaagc aggtttacca gtttgagtac gagtcactcc tgcgccgtag cgccattgct | 21540 |
| tcttcccccg accgctgtat aacgctggaa aagtccaccc aaagcgtaca ggggcccaac | 21600 |
| tcggccgtct gtggactatt ctgctgcatg tttctccacg cctttgccaa ctggccccaa | 21660 |
| actcccatgg atcacaaccc caccatgaac cttattaccg gggtacccaa ctccatgctc | 21720 |
| aacagtcccc aggtacagcc caccctgcgt cgcaaccagg aacagctcta cagcttcctg | 21780 |
| gagcgccact cgccctactt ccgcagccac agtgcgcaga ttaggagcgc cacttctttt | 21840 |
| tgtcacttga aaaacatgta aaaataatgt actagagaca ctttcaataa aggcaaatgc | 21900 |
| ttttatttgt acactctcgg gtgattattt acccccaccc ttgccgtctg cgccgtttaa | 21960 |
| aaatcaaagg ggttctgccg cgcatcgcta tgcgccactg gcagggacac gttgcgatac | 22020 |
| tggtgtttag tgctccactt aaactcaggc acaaccatcc gcggcagctc ggtgaagttt | 22080 |
| tcactccaca ggctgcgcac catcaccaac gcgtttagca ggtcgggcgc cgatatcttg | 22140 |
| aagtcgcagt tggggcctcc gccctgcgcg cgcgagttgc gatacacagg gttgcagcac | 22200 |
| tggaacacta tcagcgccgg gtggtgcacg ctggccagca cgctcttgtc ggagatcaga | 22260 |
| tccgcgtcca ggtcctccgc gttgctcagg gcgaacggag tcaactttgg tagctgcctt | 22320 |
| cccaaaaagg gcgcgtgccc aggctttgag ttgcactcgc accgtagtgg catcaaaagg | 22380 |
| tgaccgtgcc cggtctgggc gttaggatac agcgcctgca taaaagcctt gatctgctta | 22440 |
| aaagccacct gagcctttgc gccttcagag aagaacatgc cgcaagactt gccggaaaac | 22500 |
| tgattggccg gacaggccgc gtcgtgcacg cagcaccttg cgtcggtgtt ggagatctgc | 22560 |
| accacatttc ggccccaccg gttcttcacg atcctggcct tgctagactg ctccttcagc | 22620 |
| gcgcgctgcc cgttttcgct cgtcacatcc atttcaatca cgtgctcctt atttatcata | 22680 |
| atgcttccgt gtagacactt aagctcgcct tcgatctcag cgcagcggtg cagccacaac | 22740 |
| gcgcagcccg tgggctcgtg atgcttgtag gtcacctctg caaacgactg caggtacgcc | 22800 |
| tgcaggaatc gccccatcat cgtcacaaag gtcttgttgc tggtgaaggt cagctgcaac | 22860 |
| ccgcggtgct cctcgttcag ccaggtcttg catacggccg ccagagcttc cacttggtca | 22920 |
| ggcagtagtt tgaagttcgc ctttagatcg ttatccacgt ggtacttgtc catcagcgcg | 22980 |
| cgcgcagcct ccatgccctt ctcccacgca gacacgatcg gcacactcag cgggttcatc | 23040 |
| accgtaattt cactttccgc ttcgctgggc tcttcctctt cctcttgcgt ccgcatacca | 23100 |
| cgcgccactg ggtcgtcttc attcagccgc cgcactgtgc gcttacctcc tttgccatgc | 23160 |
| ttgattagca ccggtgggtt gctgaaaccc accatttgta gcgccacatc ttctctttct | 23220 |
| tcctcgctgt ccacgattac ctctggtgat ggcgggcgct cgggcttggg agaagggcgc | 23280 |
| ttctttttct tcttgggcgc aatggccaaa tccgccgccg aggtcgatgg ccgcgggctg | 23340 |
| ggtgtgcgcg gcaccagcgc gtcttgtgat gagtcttcct cgtcctcgga ctcgatacgc | 23400 |
| cgcctcatcc gcttttttgg gggcgcccgg ggaggcggcg gcgacgggga cggggacgac | 23460 |
| acgtcctcca tggttggggg acgtcgcgcc gcaccgcgtc cgcgctcggg ggtggtttcg | 23520 |
| cgctgctcct cttcccgact ggccatttcc ttctcctata ggcagaaaaa gatcatggag | 23580 |
| tcagtcgaga agaaggacag cctaaccgcc ccctctgagt tcgccaccac cgcctccacc | 23640 |
| gatgccgcca acgcgcctac caccttcccc gtcgaggcac ccccgcttga ggaggaggaa | 23700 |
| gtgattatcg agcaggaccc aggttttgta agcgaagacg acgaggaccg ctcagtacca | 23760 |
| acagaggata aaaagcaaga ccaggacaac gcagaggcaa acgaggaaca agtcgggcgg | 23820 |
| ggggacgaaa ggcatggcga ctacctagat gtgggagacg acgtgctgtt gaagcatctg | 23880 |
| cagcgccagt gcgccattat ctgcgacgcg ttgcaagagc gcagcgatgt gcccctcgcc | 23940 |
| atagcggatg tcagccttgc ctacgaacgc cacctattct caccgcgcgt accccccaaa | 24000 |
| cgccaagaaa acggcacatg cgagcccaac ccgcgcctca acttctaccc cgtatttgcc | 24060 |
| gtgccagagg tgcttgccac ctatcacatc tttttccaaa actgcaagat acccctatcc | 24120 |
| tgccgtgcca accgcagccg agcggacaag cagctggcct tgcggcaggg cgctgtcata | 24180 |
| cctgatatcg cctcgctcaa cgaagtgcca aaaatctttg agggtcttgg acgcgacgag | 24240 |
| aagcgcgcgg caaacgctct gcaacaggaa aacagcgaaa atgaaagtca ctctggagtg | 24300 |
| ttggtggaac tcgagggtga caacgcgcgc ctagccgtac taaaacgcag catcgaggtc | 24360 |
| acccactttg cctacccggc acttaaccta ccccccaagg tcatgagcac agtcatgagt | 24420 |
| gagctgatcg tgcgccgtgc gcagcccctg gagagggatg caaatttgca agaacaaaca | 24480 |
| gaggagggcc tacccgcagt tggcgacgag cagctagcgc gctggcttca aacgcgcgag | 24540 |
| cctgccgact tggaggagcg acgcaaacta atgatggccg cagtgctcgt taccgtggag | 24600 |
| cttgagtgca tgcagcggtt ctttgctgac ccggagatgc agcgcaagct agaggaaaca | 24660 |
| ttgcactaca cctttcgaca gggctacgta cgccaggcct gcaagatctc caacgtggag | 24720 |
| ctctgcaacc tggtctccta ccttggaatt ttgcacgaaa accgccttgg gcaaaacgtg | 24780 |
| cttcattcca cgctcaaggg cgaggcgcgc cgcgactacg tccgcgactg cgtttactta | 24840 |
| tttctatgct acacctggca gacggccatg ggcgtttggc agcagtgctt ggaggagtgc | 24900 |
| aacctcaagg agctgcagaa actgctaaag caaaacttga aggacctatg gacggcctcc | 24960 |
| aacgagcgct ccgtggccgc gcacctggcg gacatcattt tccccgaacg cctgcttaaa | 25020 |
| accctgcaac agggtctgcc agacttcacc agtcaaagca tgttgcagaa ctttaggaac | 25080 |
| tttatcctag agcgctcagg aatcttgccc gccacctgct gtgcacttcc tagcgacttt | 25140 |
| gtgcccatta agtaccgcga atgccctccg ccgctttggg gccactgcta ccttctgcag | 25200 |
| ctagccaact accttgccta ccactctgac ataatggaag acgtgagcgg tgacggtcta | 25260 |
| ctggagtgtc actgtcgctg caacctatgc accccgcacc gctccctggt ttgcaattcg | 25320 |
| cagctgctta acgaaagtca aattatcggt acctttgagc tgcagggtcc ctcgcctgac | 25380 |
| gaaaagtccg cggctccggg gttgaaactc actccggggc tgtggacgtc ggcttacctt | 25440 |
| cgcaaatttg tacctgagga ctaccacgcc cacgagatta ggttctacga agaccaatcc | 25500 |
| cgcccgccta atgcggagct taccgcctgc gtcattaccc agggccacat tcttggccaa | 25560 |
| ttgcaagcca tcaacaaagc ccgccaagag tttctgctac gaaagggacg gggggtttac | 25620 |
| ttggaccccc agtccggcga ggagctcaac ccaatccccc cgccgccgca gccctatcag | 25680 |
| cagcagccgc gggcccttgc ttcccaggat ggcacccaaa aagaagctgc agctgccgcc | 25740 |
| gccacccacg gacgaggagg aatactggga cagtcaggca gaggaggttt tggacgagga | 25800 |
| ggaggaggac atgatggaag actgggagag cctagacgag gaagcttccg aggtcgaaga | 25860 |
| ggtgtcagac gaaacaccgt caccctcggt cgcattcccc tcgccggcgc cccagaaatc | 25920 |
| ggcaaccggt tccagcatgg ctacaacctc cgctcctcag gcgccgccgg cactgcccgt | 25980 |
| tcgccgaccc aaccgtagat gggacaccac tggaaccagg gccggtaagt ccaagcagcc | 26040 |
| gccgccgtta gcccaagagc aacaacagcg ccaaggctac cgctcatggc gcgggcacaa | 26100 |
| gaacgccata gttgcttgct tgcaagactg tgggggcaac atctccttcg cccgccgctt | 26160 |
| tcttctctac catcacggcg tggccttccc ccgtaacatc ctgcattact accgtcatct | 26220 |
| ctacagccca tactgcaccg gcggcagcgg cagcaacagc agcggccaca cagaagcaaa | 26280 |
| ggcgaccgga tagcaagact ctgacaaagc ccaagaaatc cacagcggcg gcagcagcag | 26340 |
| gaggaggagc gctgcgtctg gcgcccaacg aacccgtatc gacccgcgag cttagaaaca | 26400 |
| ggatttttcc cactctgtat gctatatttc aacagagcag gggccaagaa caagagctga | 26460 |
| aaataaaaaa caggtctctg cgatccctca cccgcagctg cctgtatcac aaaagcgaag | 26520 |
| atcagcttcg gcgcacgctg gaagacgcgg aggctctctt cagtaaatac tgcgcgctga | 26580 |
| ctcttaagga ctagtttcgc gccctttctc aaatttaagc gcgaaaacta cgtcatctcc | 26640 |
| agcggccaca cccggcgcca gcacctgttg tcagcgccat tatgagcaag gaaattccca | 26700 |
| cgccctacat gtggagttac cagccacaaa tgggacttgc ggctggagct gcccaagact | 26760 |
| actcaacccg aataaactac atgagcgcgg gaccccacat gatatcccgg gtcaacggaa | 26820 |
| tacgcgccca ccgaaaccga attctcctgg aacaggcggc tattaccacc acacctcgta | 26880 |
| ataaccttaa tccccgtagt tggcccgctg ccctggtgta ccaggaaagt cccgctccca | 26940 |
| ccactgtggt acttcccaga gacgcccagg ccgaagttca gatgactaac tcaggggcgc | 27000 |
| agcttgcggg cggctttcgt cacagggtgc ggtcgcccgg gcagggtata actcacctga | 27060 |
| caatcagagg gcgaggtatt cagctcaacg acgagtcggt gagctcctcg cttggtctcc | 27120 |
| gtccggacgg gacatttcag atcggcggcg ccggccgctc ttcattcacg cctcgtcagg | 27180 |
| caatccaaac tctgcagacc tcgtcctctg agccgcgctc tggaggcatt ggaactctgc | 27240 |
| aatttattga ggagtttgtg ccatcggtct actttaaccc cttctcggga cctcccggcc | 27300 |
| actatccgga tcaatttatt cctaactttg acgcggtaaa ggactcggcg gacggctacg | 27360 |
| actgaatgtt ataagttcct gtccatccgc acccactatc ttcatgttgt tgcagatgaa | 27420 |
| gcgcgcaaga ccgtctgaag ataccttcaa ccccgtgtat ccatatgaca cggaaaccgg | 27480 |
| tcctccaact gtgccttttc ttactcctcc ctttgtatcc cccaatgggt ttcaagagag | 27540 |
| tccccctggg gtactctctt tgcgcctatc cgaacctcta gttacctcca atggcatgct | 27600 |
| tgcactcaaa atgggcaacg gcctctctct ggacgaggcc ggcaacctta cctcccaaaa | 27660 |
| tgtaaccact gtgagcccac ctctcaaaaa aaccaagtca aacataaacc tggaaatatc | 27720 |
| tgcacccctc acagttacct cagaagccct aactgtggct gccgccgcac ctctaatggt | 27780 |
| cgcgggcaac acactcacca tgcaatcaca ggccccgcta accgtgcacg actccaaact | 27840 |
| tagcattgcc acccaaggac ccctcacagt gtcagaagga aagctagccc tgcaaacatc | 27900 |
| aggccccctc accaccaccg atagcagtac ccttactatc actgcctcac cccctctaac | 27960 |
| tactgccact ggtagcttgg gcattgactt gaaagagccc atttatacac aaaatggaaa | 28020 |
| actaggacta aagtacgggg ctcctttgca tgtaacagac gacctaaaca ctttgaccgt | 28080 |
| agcaactggt ccaggtgtga ctattaataa tacttccttg caaactaaag ttactggagc | 28140 |
| cttgggtttt gattcacaag gcaatatgca acttaatgta gcaggaggac taaggattga | 28200 |
| ttctcaaaac agacgcctta tacttgatgt tagttatccg tttgatgctc aaaaccaact | 28260 |
| aaatctaaga ctaggacagg gccctctttt tataaactca gcccacaact tggatattaa | 28320 |
| ctacaacaaa ggcctttact tgtttacagc ttcaaacaat tccaaaaagc ttgaggttaa | 28380 |
| cctaagcact gccaaggggt tgatgtttga cgctacagcc atagccatta atgcaggaga | 28440 |
| tgggcttgaa tttggttcac ctaatgcacc aaacacaaat cccctcaaaa caaaaattgg | 28500 |
| ccatggccta gaatttgatt caaacaaggc tatggttcct aaactaggaa ctggccttag | 28560 |
| ttttgacagc acaggtgcca ttacagtagg aaacaaaaat aatgataagc taactttgtg | 28620 |
| gaccacacca gctccatctc ctaactgtag actaaatgca gagaaagatg ctaaactcac | 28680 |
| tttggtctta acaaaatgtg gcagtcaaat acttgctaca gtttcagttt tggctgttaa | 28740 |
| aggcagtttg gctccaatat ctggaacagt tcaaagtgct catcttatta taagatttga | 28800 |
| cgaaaatgga gtgctactaa acaattcctt cctggaccca gaatattgga actttagaaa | 28860 |
| tggagatctt actgaaggca cagcctatac aaacgctgtt ggatttatgc ctaacctatc | 28920 |
| agcttatcca aaatctcacg gtaaaactgc caaaagtaac attgtcagtc aagtttactt | 28980 |
| aaacggagac aaaactaaac ctgtaacact aaccattaca ctaaacggta cacaggaaac | 29040 |
| aggagacaca actccaagtg catactctat gtcattttca tgggactggt ctggccacaa | 29100 |
| ctacattaat gaaatatttg ccacatcctc ttacactttt tcatacattg cccaagaata | 29160 |
| aagaatcgtt tgtgttatgt ttcaacgtgt ttatttttca attgcagaaa atttcaagtc | 29220 |
| atttttcatt cagtagtata gccccaccac cacatagctt atacagatca ccgtacctta | 29280 |
| atcaaactca cagaacccta gtattcaacc tgccacctcc ctcccaacac acagagtaca | 29340 |
| cagtcctttc tccccggctg gccttaaaaa gcatcatatc atgggtaaca gacatattct | 29400 |
| taggtgttat attccacacg gtttcctgtc gagccaaacg ctcatcagtg atattaataa | 29460 |
| actccccggg cagctcactt aagttcatgt cgctgtccag ctgctgagcc acaggctgct | 29520 |
| gtccaacttg cggttgctta acgggcggcg aaggagaagt ccacgcctac atgggggtag | 29580 |
| agtcataatc gtgcatcagg atagggcggt ggtgctgcag cagcgcgcga ataaactgct | 29640 |
| gccgccgccg ctccgtcctg caggaataca acatggcagt ggtctcctca gcgatgattc | 29700 |
| gcaccgcccg cagcataagg cgccttgtcc tccgggcaca gcagcgcacc ctgatctcac | 29760 |
| ttaaatcagc acagtaactg cagcacagca ccacaatatt gttcaaaatc ccacagtgca | 29820 |
| aggcgctgta tccaaagctc atggcgggga ccacagaacc cacgtggcca tcataccaca | 29880 |
| agcgcaggta gattaagtgg cgacccctca taaacacgct ggacataaac attacctctt | 29940 |
| ttggcatgtt gtaattcacc acctcccggt accatataaa cctctgatta aacatggcgc | 30000 |
| catccaccac catcctaaac cagctggcca aaacctgccc gccggctata cactgcaggg | 30060 |
| aaccgggact ggaacaatga cagtggagag cccaggactc gtaaccatgg atcatcatgc | 30120 |
| tcgtcatgat atcaatgttg gcacaacaca ggcacacgtg catacacttc ctcaggatta | 30180 |
| caagctcctc ccgcgttaga accatatccc agggaacaac ccattcctga atcagcgtaa | 30240 |
| atcccacact gcagggaaga cctcgcacgt aactcacgtt gtgcattgtc aaagtgttac | 30300 |
| attcgggcag cagcggatga tcctccagta tggtagcgcg ggtttctgtc tcaaaaggag | 30360 |
| gtagacgatc cctactgtac ggagtgcgcc gagacaaccg agatcgtgtt ggtcgtagtg | 30420 |
| tcatgccaaa tggaacgccg gacgtagtca tatttcctga agcaaaacca ggtgcgggcg | 30480 |
| tgacaaacag atctgcgtct ccggtctcgc cgcttagatc gctctgtgta gtagttgtag | 30540 |
| tatatccact ctctcaaagc atccaggcgc cccctggctt cgggttctat gtaaactcct | 30600 |
| tcatgcgccg ctgccctgat aacatccacc accgcagaat aagccacacc cagccaacct | 30660 |
| acacattcgt tctgcgagtc acacacggga ggagcgggaa gagctggaag aaccatgttt | 30720 |
| ttttttttat tccaaaagat tatccaaaac ctcaaaatga agatctatta agtgaacgcg | 30780 |
| ctcccctccg gtggcgtggt caaactctac agccaaagaa cagataatgg catttgtaag | 30840 |
| atgttgcaca atggcttcca aaaggcaaac ggccctcacg tccaagtgga cgtaaaggct | 30900 |
| aaacccttca gggtgaatct cctctataaa cattccagca ccttcaacca tgcccaaata | 30960 |
| attctcatct cgccaccttc tcaatatatc tctaagcaaa tcccgaatat taagtccggc | 31020 |
| cattgtaaaa atctgctcca gagcgccctc caccttcagc ctcaagcagc gaatcatgat | 31080 |
| tgcaaaaatt caggttcctc acagacctgt ataagattca aaagcggaac attaacaaaa | 31140 |
| ataccgcgat cccgtaggtc ccttcgcagg gccagctgaa cataatcgtg caggtctgca | 31200 |
| cggaccagcg cggccacttc cccgccagga accatgacaa aagaacccac actgattatg | 31260 |
| acacgcatac tcggagctat gctaaccagc gtagccccga tgtaagcttg ttgcatgggc | 31320 |
| ggcgatataa aatgcaaggt gctgctcaaa aaatcaggca aagcctcgcg caaaaaagaa | 31380 |
| agcacatcgt agtcatgctc atgcagataa aggcaggtaa gctccggaac caccacagaa | 31440 |
| aaagacacca tttttctctc aaacatgtct gcgggtttct gcataaacac aaaataaaat | 31500 |
| aacaaaaaaa catttaaaca ttagaagcct gtcttacaac aggaaaaaca acccttataa | 31560 |
| gcataagacg gactacggcc atgccggcgt gaccgtaaaa aaactggtca ccgtgattaa | 31620 |
| aaagcaccac cgacagctcc tcggtcatgt ccggagtcat aatgtaagac tcggtaaaca | 31680 |
| catcaggttg attcacatcg gtcagtgcta aaaagcgacc gaaatagccc gggggaatac | 31740 |
| atacccgcag gcgtagagac aacattacag cccccatagg aggtataaca aaattaatag | 31800 |
| gagagaaaaa cacataaaca cctgaaaaac cctcctgcct aggcaaaata gcaccctccc | 31860 |
| gctccagaac aacatacagc gcttccacag cggcagccat aacagtcagc cttaccagta | 31920 |
| aaaaagaaaa cctattaaaa aaacaccact cgacacggca ccagctcaat cagtcacagt | 31980 |
| gtaaaaaagg gccaagtgca gagcgagtat atataggact aaaaaatgac gtaacggtta | 32040 |
| aagtccacaa aaaacaccca gaaaaccgca cgcgaaccta cgcccagaaa cgaaagccaa | 32100 |
| aaaacccaca acttcctcaa atcgtcactt ccgttttccc acgttacgtc acttcccatt | 32160 |
| ttaagaaaac tacaattccc aacacataca agttactccg ccctaaaacc tacgtcaccc | 32220 |
| gccccgttcc cacgccccgc gccacgtcac aaactccacc ccctcattat catattggct | 32280 |
| tcaatccaaa ataaggtata ttattgatga tgttacatcg ttaattaacg atttcgaacc | 32340 |
| cggggtaccg aattcctcga gtctagagga gcatgcgacg tcgcaattcg ccctatagtg | 32400 |
| agtcgtatta caattcactg gccgtcgttt tacaacgtcg tgactgggaa aaccctggcg | 32460 |
| ttacccaact taatcgcctt gcagcacatc cccctttcgc cagctggcgt aatagcgaag | 32520 |
| aggcccgcac cgatcgccct tcccaacagt tgcgcagcct gaatggcgaa tggaaattgt | 32580 |
| aagcgttaat attttgttaa aattcgcgtt aaatttttgt taaatcagct cattttttaa | 32640 |
| ccaataggcc gaaatcggca aaatccctta taaatcaaaa gaatagaccg agatagggtt | 32700 |
| gagtgttgtt ccagtttgga acaagagtcc actattaaag aacgtggact ccaacgtcaa | 32760 |
| agggcgaaaa accgtctatc agggcgatgg cccactacgt gaaccatcac cctaatcaag | 32820 |
| ttttttgggg tcgaggtgcc gtaaagcact aaatcggaac cctaaaggga gcccccgatt | 32880 |
| tagagcttga cggggaaagc cggcgaacgt ggcgagaaag gaagggaaga aagcgaaagg | 32940 |
| agcgggcgct agggcgctgg caagtgtagc ggtcacgctg cgcgtaacca ccacacccgc | 33000 |
| cgcgcttaat gcgccgctac agggcgcgtc ctgatgcggt attttctcct tacgcatctg | 33060 |
| tgcggtattt cacaccgcat acaggtggca cttttcgggg aaatgtgcgc ggaaccccta | 33120 |
| tttgtttatt tttctaaata cattcaaata tgtatccgct catgagacaa taaccctgat | 33180 |
| aaatgcttca ataatattga aaaaggaaga gtatgagtat tcaacatttc cgtgtcgccc | 33240 |
| ttattccctt ttttgcggca ttttgccttc ctgtttttgc tcacccagaa acgctggtga | 33300 |
| aagtaaaaga tgctgaagat cagttgggtg cacgagtggg ttacatcgaa ctggatctca | 33360 |
| acagcggtaa gatccttgag agttttcgcc ccgaagaacg ttttccaatg atgagcactt | 33420 |
| ttaaagttct gctatgtggc gcggtattat cccgtattga cgccgggcaa gagcaactcg | 33480 |
| gtcgccgcat acactattct cagaatgact tggttgagta ctcaccagtc acagaaaagc | 33540 |
| atcttacgga tggcatgaca gtaagagaat tatgcagtgc tgccataacc atgagtgata | 33600 |
| acactgcggc caacttactt ctgacaacga tcggaggacc gaaggagcta accgcttttt | 33660 |
| tgcacaacat gggggatcat gtaactcgcc ttgatcgttg ggaaccggag ctgaatgaag | 33720 |
| ccataccaaa cgacgagcgt gacaccacga tgcctgtagc aatggcaaca acgttgcgca | 33780 |
| aactattaac tggcgaacta cttactctag cttcccggca acaattaata gactggatgg | 33840 |
| aggcggataa agttgcagga ccacttctgc gctcggccct tccggctggc tggtttattg | 33900 |
| ctgataaatc tggagccggt gagcgtgggt ctcgcggtat cattgcagca ctggggccag | 33960 |
| atggtaagcc ctcccgtatc gtagttatct acacgacggg gagtcaggca actatggatg | 34020 |
| aacgaaatag acagatcgct gagataggtg cctcactgat taagcattgg taactgtcag | 34080 |
| accaagttta ctcatatata ctttagattg atttaaaact tcatttttaa tttaaaagga | 34140 |
| tctaggtgaa gatccttttt gataatctca tgaccaaaat cccttaacgt gagttttcgt | 34200 |
| tccactgagc gtcagacccc gtagaaaaga tcaaaggatc ttcttgagat cctttttttc | 34260 |
| tgcgcgtaat ctgctgcttg caaacaaaaa aaccaccgct accagcggtg gtttgtttgc | 34320 |
| cggatcaaga gctaccaact ctttttccga aggtaactgg cttcagcaga gcgcagatac | 34380 |
| caaatactgt tcttctagtg tagccgtagt taggccacca cttcaagaac tctgtagcac | 34440 |
| cgcctacata cctcgctctg ctaatcctgt taccagtggc tgctgccagt ggcgataagt | 34500 |
| cgtgtcttac cgggttggac tcaagacgat agttaccgga taaggcgcag cggtcgggct | 34560 |
| gaacgggggg ttcgtgcaca cagcccagct tggagcgaac gacctacacc gaactgagat | 34620 |
| acctacagcg tgagctatga gaaagcgcca cgcttcccga agggagaaag gcggacaggt | 34680 |
| atccggtaag cggcagggtc ggaacaggag agcgcacgag ggagcttcca gggggaaacg | 34740 |
| cctggtatct ttatagtcct gtcgggtttc gccacctctg acttgagcgt cgatttttgt | 34800 |
| gatgctcgtc aggggggcgg agcctatgga aaaacgccag caacgcggcc tttttacggt | 34860 |
| tcctggcctt ttgctggcct tttgctcaca tgttctttcc tgcgttatcc cctgattctg | 34920 |
| tggataaccg tattaccgcc tttgagtgag ctgataccgc tcgccgcagc cgaacgaccg | 34980 |
| agcgcagcga gtcagtgagc gaggaagcgg aagagcgccc aatacgcaaa ccgcctctcc | 35040 |
| ccgcgcgttg gccgattcat taatgcagct ggcacgacag gtttcccgac tggaaagcgg | 35100 |
| gcagtgagcg caacgcaatt aatgtgagtt agctcactca ttaggcaccc caggctttac | 35160 |
| actttatgct tccggctcgt atgttgtgtg gaattgtgag cggataacaa tttcacacag | 35220 |
| gaaacagcta tgaccatgat tacgccaagc tatttaggtg acactataga atactcaagc | 35280 |
| tagttaatta acgttaatta acatcatcaa taatatacct tattttggat tgaagccaat | 35340 |
| atgataatga gggggtggag tttgtgacgt ggcgcggggc gtgggaacgg ggcgggtgac | 35400 |
| gtagtagtgt ggcggaagtg tgatgttgca agtgtggcgg aacacatgta agcgacggat | 35460 |
| gtggcaaaag tgacgttttt ggtgtgcgcc ggtgtacaca ggaagtgaca attttcgcgc | 35520 |
| ggttttaggc ggatgttgta gtaaatttgg gcgtaaccga gtaagatttg gccattttcg | 35580 |
| cgggaaaact gaataagagg aagtgaaatc tgaacaattt tgtgttactc atagcgcgta | 35640 |
| atctctagca t | 35651 |

| <212> | Type: | DNA |
|---|---|---|
| <211> | Length: | 35651 |
| | SequenceName: | SEQ ID No. 1 |
| | SequenceDescription: | Custom Codon |
| ------------ | | |
| | Sequence Name: | SEQ ID No.1 |

### REFERENCES

1. Andersen, M. M. and T. Ronne. 1991. Side-effects with Japanese encephalitis vaccine. Lancet 337:1044.
2. Ashok, M. S. and P. N. Rangarajan. 2000. Immunization with plasmid DNA encoding the envelope glycoprotein of Japanese encephalitis virus confers significant protection against intracerebral viral challenge without inducing detectable antiviral antibodies. Vaccine 18:68-75.
3. Both, G. W., L. J. Lockett, V. Janardhana, S. J. Edwards, A. R. Bellamy, F. L. Graham, L. Prevec, and M. E. Andrew. 1993. Protective immunity to rotavirus-induced diarrhea is passively transferred to newborn mice from naive dams vaccinated with a single dose of a recombinant adenovirus expressing rotavirus VP7sc. Virology 193:940-950.
4. Casimiro, D. R., A. Tang, L. Chen, T. M. Fu, R. K. Evans, M. E. Davies, D. C. Freed, W. Hurni, J. M. Aste-Amezaga, L. Guan, R. Long, L. Huang, V. Harris, D. K. Nawrocki, H. Mach, R. D. Troutman, L. A. Isopi, K. K. Murthy, K. Rice, K. A. Wilson, D. B. Volkin, E. A. Emini, and J. W. Shiver. 2003. Vaccine-induced immunity in baboons by using DNA and replication-incompetent adenovirus type 5 vectors expressing a human immunodeficiency virus type 1 gag gene. J Virol. 77:7663-7668.
5. Chambers, T. J., C. S. Hahn, R. Galler, and C. M. Rice. 1990. Flavivirus genome organization, expression and replication. Annu. Rev. Microbiol. 44:649.
6. Chang, G.-J. J., A. R. Hunt, and B. Davis. 2000. A single intramuscular injection of recombinant plasmid DNA induces protective immunity and prevents Japanese encephalitis in mice. Journal of Virology 74:4244-4252.
7. Chen, H. W., C. H. Pan, M. Y. Liau, R. Jou, C. J. Tsai, H. J. Wu, Y. L. Lin, and M. H. Tao. 1999. Screening of protective antigens of Japanese encephalitis virus by DNA immunization: a comparative study with conventional viral vaccines. J. Virol. 73:10137-10145.
8. Chengalvala, M. V., B. M. Bhat, R. A. Bhat, S. K. Dheer, M. D. Lubeck, R. H. Purcell, and K. K. Murthy. 1997. Replication and immunogenicity of Ad7-, Ad4-, and Ad5-hepatitis B virus surface antigen recombinants, with or without a portion of E3 region, in chimpanzees. Vaccine 15:335-339.
9. Flanagan, B., C. R. Pringle, and K. N. Leppard. 1997. A recombinant human adenovirus expressing the simian immunodeficiency virus Gag antigen can induce long-lived immune responses in mice. J Gen. Virol. 78 (Pt 5):991-997.
10. Fonseca, B. A., S. Pincus, R. E. Shope, E. Paoletti, and P. W. Mason. 1994. Recombinant vaccinia viruses co-expressing dengue-1 glycoproteins prM and E induce neutralizing antibodies in mice. Vaccine 12:279-285.
11. Fooks, A. R., D. Jeevarajah, J. Lee, A. Warnes, S. Niewiesk, M. ter, V, J. R. Stephenson, and J. C. Clegg. 1998. Oral or parenteral administration of replication-deficient adenoviruses expressing the measles virus hemagglutinin and fusion proteins: protective immune responses in rodents. J Gen. Virol. 79 (Pt 5):1027-1031.
12. Guirakhoo, F., R. Weltzin, T. J. Chambers, Z. X. Zhang, K. Soike, M. Ratterree, J. Arroyo, K. Georgakopoulos, J. Catalan, and T. P. Monath. 2000. Recombinant chimeric yellow fever-dengue type 2 virus is immunogenic and protective in nonhuman primates. J Virol. 74:5477-5485.
13. Imler J. L. 1995. Adenovirus vectors as recombinant viral vaccines. Vaccine 13:1143-1151.
14. Jan, L. R., C. S. Yang, L. S. Henchal, H. Sumiyoshi, P. L. Summers, D. R. Dubois, and C. J. Lai. 1993. Increased immunogenicity and protective efficacy in outbred and inbred mice by strategic carboxyl-terminal truncation of Japanese encephalitis virus envelope glycoprotein. Am. J Trop. Med. Hyg. 48:412-423.
15. Kaur, R., G. Sachdeva, and S. Vrati. 2002. Plasmid DNA immunization against Japanese Encephalitis Virus: immunogenicity of membrane-anchored and secretory envelope protein. J. Infect. Dis. 185:1-12.
16. Kaur, R. and S. Vrati. 2003. Development of a recombinant vaccine against Japanese encephalitis. J Neurovirol 9:421-431.
17. Kimura-Kiroda, J. and K. Yasui. 1988. Protection of mice against Japanese encephalitis virus by passive administration of monoclonal antibodies. J. Immunol. 141:3606-3610.
18. Konishi, E., S. Pincus, E. Paoletti, R. E. Shope, T. Burrage, and P. W. Mason. 1992. Mice immunized with a subviral particle containing the Japanese encephalitis virus prM/M and E proteins are protected from lethal JEV infection. Virology 188:714-720.
19. Konishi, E., M. Yamaoka, S. W. Khin, I. Kurane, K. Takada, and P. W. Mason. 1999. The anamnestic neutralizing antibody response is critical for protection of mice from challenge following vaccination with a plasmid encoding the Japanese encephalitis virus premembrane and envelope genes. J. Virol. 73:5527-5534.
20. Konishi, E., M. Yamaoka, Khin-Sane-Win, I. Kurane, and Mason P. W. 1998. Induction of protective immunity against Japanese encephalitis in mice by immunization with a plasmid encoding Japanese encephalitis virus premembrane and envelope genes. Journal of Virology 72:4925-4930.
21. Lemiale, F., W. P. Kong, L. M. Akyurek, X. Ling, Y. Huang, B. K. Chakrabarti, M. Eckhaus, and G. J. Nabel. 2003. Enhanced mucosal immunoglobulin A response of intranasal adenoviral vector human immunodeficiency virus vaccine and localization in the central nervous system. J Virol. 77:10078-10087.
22. Lin; B., C. R. Parrish, J. M. Murray, and P. J. Wright. 1994. Localization of a neutralizing epitope on the envelope protein of dengue virus type 2. Virology 202:885-890.
23. Livingston, P. G., I. Kurane, C. J. Lai, M. Bray, and F. A. Ennis. 1994. Recognition of envelope protein by dengue virus serotype-specific human CD4+ CD8− cytotoxic T-cell clones. J Virol. 68:3283-3288.
24. Lubeck, M. D., A. R. Davis, M. Chengalvala, R. J. Natuk, J. E. Morin, K. Molnar-Kimber, B. B. Mason, B. M. Bhat, S. Mizutani, P. P. Hung, and 1989. Immunogenicity and efficacy testing in chimpanzees of an oral hepatitis B vaccine based on live recombinant adenovirus. Proc. Natl. Acad. Sci. U.S.A 86:6763-6767.
25. Mason P. W., S. Pincus, M. J. Fournier, T. L. Mason, R. E. Shope, and E. Paoletti. 1991. Japanese encephalitis virus-Vaccinia recombinants produce particulate forms of the structural membrane proteins and induce high levels of protection against lethal JEV infection. Virology 180:294-305.
26. McMinn, P. C. 1997. The molecular basis of virulence of the encephalitogenic flaviviruses. J Gen. Virol. 78 (Pt 11):2711-2722.
27. Men, R., M. Bray, and C.-J. Lai. 1991. Carboxy-terminal truncated dengue virus envelope glycoprotein expressed on the cell surface and secreted intracellularly exhibit increased immunogenicity in mice. Journal of Virology 65:1400-1407.
28. Monath, T. P. 2001. Prospects for development of a vaccine against the West Nile virus. Ann. N.Y. Acad. Sci. 951:1-12.
29. Monath, T. P. 2002. Japanese encephalitis vaccines: current vaccines and future prospects. Curr. Top. Microbiol. Immunol. 267:105-138.
30. Monath, T. P., K. McCarthy, P. Bedford, C. T. Johnson, R. Nichols, S. Yoksan, R. Marchesani, M. Knauber, K. H. Wells, J. Arroyo, and F. Guirakhoo. 2002. Clinical proof of principle for ChimeriVax: recombinant live, attenuated vaccines against flavivirus infections. Vaccine 20:1004-1018.
31. Monath, T. P., K. Soike, I. Levenbook, Z. X. Zhang, J. Arroyo, S. Delagrave, G. Myers, A. D. Barrett, R. E. Shope, M. Ratterree, T. J. Chambers, and F. Guirakhoo. 1999. Recombinant, chimaeric live, attenuated vaccine (ChimeriVax) incorporating the envelope genes of Japanese encephalitis (SA14-14-2) virus and the capsid and nonstructural genes of yellow fever (17D) virus is safe, immunogenic and protective in non-human primates. Vaccine 17:1869-1882.
32. Pan, C. H., H. W. Chen, H. W. Huang, and M. H. Tao. 2001. Protective mechanisms induced by a Japanese encephalitis virus DNA vaccine: requirement for antibody but not CD8(+) cytotoxic T-cell responses. J Virol. 75:11457-11463.
33. Plesner, A. M. and T. Ronne. 1997. Allergic mucocutaneous reactions to Japanese encephalitis vaccine. Vaccine 15:1239-1243.
34. Putnak, R., R. Feighny, J. Burrous, M. Cochran, C. Hackett, G. Smith, and C. Hoke. 1991. Dengue-1 virus envelope glycoprotein gene expressed in recombinant baculovirus elicits virus-neutralizing antibody in mice and protects them from virus challenge. Am. J Trop. Med. Hyg. 45:159-167.
35. Raikwar, S. P., P. Malik, O. Singh, and S. Vrati. 1997. Recombinant adenovirus synthesizing cell surface-anchored beta hCG induces bioneutralizing antibodies in rats. Gene 190:197-202.
36. Ramakrishna, C., A. Desai, S. K. Shankar, A. Chandramuki, and V. Ravi. 1999. Oral immunisation of mice with live Japanese encephalitis virus induces a protective immune response. Vaccine 17:3102-3108.
37. Raviprakash, K., T. J. Kochel, D. Ewing, M. Simmons, I. Phillips, C. G. Hayes, and K. R. Porter. 2000. Immunogenicity of dengue virus type 1 DNA vaccines expressing truncated and full length envelope protein. Vaccine 18:2426-2434.
38. Ruff, T. A., D. Eisen, A. Fuller, and R. Kas. 1991. Adverse reactions to Japanese encephalitis vaccine. Lancet 338:881-882.
39. Sakaguchi, M., M. Yoshida, W. Kuroda, O. Harayama, Y. Matsunaga, and S. Inouye. 2000. Systemic immediate-type reactions to gelatin included in Japanese encephalitis vaccines. Vaccine 15:121-122.
40. Sharpe, S., A. Fooks, J. Lee, K. Hayes, C. Clegg, and M. Cranage. 2002. Single oral immunization with replication deficient recombinant adenovirus elicits long-lived transgene-specific cellular and humoral immune responses. Virology 293:210-216.
41. Shiver, J. W., T. M. Fu, L. Chen, D. R. Casimiro, M. E. Davies, R. K. Evans, Z. Q. Zhang, A. J. Simon, W. L. Trigona, S. A. Dubey, L. Huang, V. A. Harris, R. S. Long, X. Liang, L. Handt, W. A. Schleif, L. Zhu, D. C. Freed, N. V. Persaud, L. Guan, K. S. Punt, A. Tang, M. Chen, K. A. Wilson, K. B. Collins, G. J. Heidecker, V. R. Fernandez, H. C. Perry, J. G. Joyce, K. M. Grimm, J. C. Cook, P. M. Keller, D. S. Kresock, H. Mach, R. D. Troutman, L. A. Isopi, D. M. Williams, Z. Xu, K. E. Bohannon, D. B. Volkin, D. C. Montefiori, A. Miura, G. R. Krivulka, M. A. Lifton, M. J. Kuroda, J. E. Schmitz, N. L. Letvin, M. J. Caulfield, A. J. Bett, R. Youil, D. C. Kaslow, and E. A. Emini. 2002. Replication-incompetent adenoviral vaccine vector elicits effective anti-immunodeficiency-virus immunity. Nature 415:331-335.
42. Vos, A., A. Neubert, E. Pommerening, T. Muller, L. Dohner, L. Neubert, and K. Hughes. 2001. Immunogenicity of an E1-deleted recombinant human adenovirus against rabies by different routes of administration. J Gen. Virol. 82:2191-2197.
43. Vrati, S., V. Agarwal, P. Malik, S. A. Wani, and M. Saini. 1999. Molecular characterization of an Indian isolate of Japanese encephalitis virus that shows an extended lag phase during growth. J. Gen. Virol. 80:1665-1671.

1. A method for protecting a subject against Japanese encephalitis virus infection comprising administering a recombinant adenovirus (RAdEs) vaccine (ECACC Accession Number 04121701) to subject in need thereof.

2. The method according to claim 1, wherein the vaccine is prepared by a process comprising the steps of:
a) digesting plasmid pMEs from Japanese encephalitis virus with restriction enzymes Kpn I and Bam HI to obtain cDNA encoding JEV proteins prM and Es,
b) ligating the cDNA to adenovirus shuttle plasmid pShuttle digested with restriction enzymes Kpn I and Hind III at the Kpn I end,
c) adding nucleotides at the free Bam HI and Hind III ends with T4 DNA polymerase to create blunt ends,
d) ligating the blunt ends together to yield shuttle plasmid pSEs with JEV cDNA encoding the proteins prM and Es,
e) digesting the shuttle plasmid pSEs with restriction enzymes IC-eu I and Pl-Sce I to obtain expression cassette containing the JEV cDNA together with the CMV promoter/enhancer and BGH polyadenylation signal,
f) ligating the digested shuttle plasmid with IC-eu I and Pl-Sce I digested adenovirus plasmid pAdeno-X to generate plasmid pAdEs containing Es expression cassette,
g) digesting the plasmid pAdEs with Pac I,
h) transfecting the monolayers HEK 293 cells with digested plasmid pAdEs for about one week, and
i) obtaining the recombinant virus RAdEs vaccine.

3. A method of protecting a subject against Japanese encephalitis virus infection comprising administering a recombinant adenovirus (RAdEs) vaccine (ECACC Accession Number 04121701), comprising JEV Es protein optionally with pharmaceutically acceptable additives to a subject in need thereof.

4. The method according to claim 1, to protect the subject from encephalitis.

5. The method according to claim 3, to protect the subject from encephalitis.

6. The method according to claim 1, wherein the subject is an animal or human.

7. The method according to claim 2, wherein the subject is an animal or human.

8. The method according to claim 3, wherein the subject is an animal or human.

9. The method according to claim 1, wherein the vaccine activates both humoral and cell-mediated immune response.

10. The method according to claim 2, wherein the vaccine activates both humoral and cell-mediated immune response.

11. The method according to claim 3, wherein the vaccine activates both humoral and cell-mediated immune response.

12. The method according to claim 9, wherein the humoral response to the vaccine comprises IgG1 type of antibody.

13. The method according to claim 10, wherein the humoral response to the vaccine comprises IgG1 type of antibody.

14. The method according to claim 11 wherein the humoral response to the vaccine comprises IgG1 type of antibody.

15. The method according to claim 1, wherein the vaccine leads to high amount of IFN-gamma secretion.

16. The method according to claim 3, wherein the vaccine leads to high amount of IFN-gamma secretion.

17. The method according to claim 1, wherein the vaccine leads to moderate levels of IL-5 synthesis.

18. The method according to claim 3, wherein the vaccine leads to moderate levels of IL-5 synthesis.

19. The method according to claim 1, wherein increased amount of the vaccine leads to higher immune response.

20. The method according to claim 3, wherein increased amount of the vaccine leads to higher immune response.

21. The method according to claim 1, wherein the vaccine is administered intra-muscularly.

22. The method according to claim 2, wherein the vaccine is administered intra-muscularly.

23. The method according to claim 3, wherein the vaccine is administered intra-muscularly.


SEQUENCE LISTING (1 sequence)

SEQ ID NO: 1 - DNA, 35651 bp, ARTIFICIAL SEQUENCE
  Custom Codon
cgtaactataacggtcctaaggtagcgaaagctcagatctggatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggatgatccgagctcggtaccatgtggctcgcaagcttggcagttgtcatagcttgcgcaggagccatgaagttgtcgaatttccaggggaagcttttgatgaccatcaacaacacggacattgcagacgttatcgtgattcccacctcaaaaggagagaacagatgctgggtccgggcaatcgacgtcggttacatgtgtgaggacaccatcacgtacgaatgtcctaagcttactgcgggcaatgatccagaggacgtggattgctggtgtgacaaccaagaagtctacgtccaatatggacggtgcacgcggaccaggcattccaagcgaagcaggagatccgtgtcggtccagacacatggggagagttcactagtgaataaaaaagaggcttggctggattcaacgaaagccacacgatatctcacgaaaactgagaactggatcataaggaatcctggctatgctttcttggcggcggtacttggctggatgcttggcagtaacaacggtcaacgcgtggtatttaccatcctcctgctgttggtcgctccggcttacagttttaattgtctgggaatgggcaatcgcgacttcatagaaggagccagtggagccacttgggtggacttggtgctagaaggagatagctgtttgacaatcatggcaaacgataaaccaacattggatgtccgcatgattaacatcgaagctagccaacttgctgaggtcagaagttattgctatcatgcctcagtcactgacatctcgacggtggctcggtgccccatgactggagaagctcacaacgagaagcgagctgatagtagctatgtgtgcaaacaaggcttcactgatcgtgggtggggcaacggatgtggacttttcgggaagggaagcattgacacatgtgcaaaattctcctgcaccagcaaagcgattgggagaacaatccagccagaaaacatcaaatacgaagttggcatttttgtgcatggaaccaccacttcggaaaaccatgggaattactcagcgcaagttggggcgtcccaggcggcaaagtttaccgtaacacccaatgctccttcgataaccctcaaacttggtgactacggagaagtcacactggactgtgagccaaggagtggactgaacactgaagcgttttacgtcatgaccgtggggtcaaagtcatttctggtccatagggaatggtttcatgacctcgctctcccctggacgtccccttcgagcacagcgtggagaaacagagaactcctcatggaatttgaagaggcgcacgccacaaaacagtccgttgttgctcttggttcacaggaaggaggcctccatcaggcgttggcaggagccatcgtggtggagtactcaagctcagtgaagttaacatcaggccacctgaaatgtaggctgaaaatggacaaactagctctgaaaggcacaacctatggcatgtgtacagaaaaattctcgttcgcgaaaaatccggcggacactggtcacggaacagttgtcattgaactctcctactctgggagtgatggcccctgcaaaattccgattgtctccgtcgcgagcctcaatgacatgactcctgttgggcggctggtgacagtgaacccctttgtcgcggcttccagtgccaactcaaaggtgctggtcgagatggaaccccccttcggagactcctatatcgtggttggaaggggagacaagcagatcaaccaccattggcacaaataataaaggatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagcagatctgcagatctgaattcatctatgtcgggtgcggagaaagaggtaatgaaatggcatcgactcgaagatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttataagttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttacatcgttaattaacgatttcgaacccggggtaccgaattcctcgagtctagaggagcatgcgacgtcgcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctagttaattaacgttaattaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatctctagcat